SEQUENCE LISTING (10 sequences)

SEQ ID NO: 1 - DNA, 32 bp, Artificial Sequence
  Synthetic polynucleotide
  modified_base (8)..(8): LNA
  modified_base (11)..(11): LNA
  modified_base (14)..(14): LNA
  modified_base (17)..(17): LNA
  modified_base (20)..(20): LNA
  modified_base (23)..(23): LNA
cgcgatcgcctgcccctgctcagaggatcgcg

SEQ ID NO: 2 - DNA, 32 bp, Artificial Sequence
  Synthetic polynucleotide
  modified_base (8)..(8): LNA
  modified_base (11)..(11): LNA
  modified_base (14)..(14): LNA
  modified_base (17)..(17): LNA
  modified_base (20)..(20): LNA
  modified_base (23)..(23): LNA
cgcgatcggacctgaccagcaaccggatcgcg

SEQ ID NO: 3 - DNA, 57 bp, Artificial Sequence
  Synthetic polynucleotide
  modified_base (4)..(4): LNA
  modified_base (8)..(8): LNA
  modified_base (11)..(11): LNA
  modified_base (14)..(14): LNA
gcctgcccctgctcagagcaatctccgccactctgagcaggacatcccacttacacc

SEQ ID NO: 4 - DNA, 40 bp, Artificial Sequence
  Synthetic polynucleotide
cagagtggcggagattgctctgagcaggcaatctccgcca

SEQ ID NO: 5 - DNA, 16 bp, Artificial Sequence
  Synthetic oligonucleotide
acatcccacttacacc

SEQ ID NO: 6 - DNA, 22 bp, Artificial Sequence
  Synthetic oligonucleotide
  modified_base (2)..(2): LNA
  modified_base (5)..(5): LNA
  modified_base (8)..(8): LNA
  modified_base (11)..(11): LNA
  modified_base (14)..(14): LNA
ggtgtaagtgggatgtcctgct

SEQ ID NO: 7 - DNA, 57 bp, Artificial Sequence
  Synthetic polynucleotide
  modified_base (4)..(4): LNA
  modified_base (8)..(8): LNA
  modified_base (11)..(11): LNA
  modified_base (14)..(14): LNA
ggacctgaccagcaaccgaccctcaatcaacggttgctggtaacttatactacctcc

SEQ ID NO: 8 - DNA, 40 bp, Artificial Sequence
  Synthetic polynucleotide
aaccgttgattgagggtcggttgctggtaccctcaatcaa

SEQ ID NO: 9 - DNA, 16 bp, Artificial Sequence
  Synthetic oligonucleotide
aacttatactacctcc

SEQ ID NO: 10 - DNA, 22 bp, Artificial Sequence
  Synthetic oligonucleotide
  modified_base (2)..(2): LNA
  modified_base (5)..(5): LNA
  modified_base (8)..(8): LNA
  modified_base (11)..(11): LNA
  modified_base (14)..(14): LNA
ggaggtagtataagttaccagc

(19) United States
(12) Patent Application Publication
(10) Pub. No.: US 2018/0179577 A1
(43) Pub. Date: Jun. 28, 2018

(54) Lipid-polymer Hybrid Nanoparticle Biochip and Application Thereof

(71) Applicants: Jiaming Hu, Arcadia, CA (US); Yan Sheng, Yantai (CN); Ly James Lee, Columbus, OH (US)

(72) Inventors: Jiaming Hu, Arcadia, CA (US); Yan Sheng, Yantai (CN); Ly James Lee, Columbus, OH (US)

(21) Appl. No.: 15/831,998

(22) Filed: Dec. 5, 2017

Related U.S. Application Data
(60) Provisional application No. 62/438,063, filed on Dec. 22, 2016.

(51) Int. Cl. C12Q 1/68

(52) U.S. Cl. CPC C12Q 1/682; C12Q 1/6886

(57) ABSTRACT

The present invention provides a novel lipid-polymer hybrid nanoparticle (LPHN) biochip. The LPHN biochip comprises a gold coating substrate with a surface layer on the gold coating and a nanoparticle, wherein the nanoparticle anchors on the surface layer and encapsulates labeling moieties which comprise molecular beacons (MB), Toehold-initiated molecular beacons (Ti-MB), biomolecules or components for performing catalyzed hairpin DNA circuit (CHDC), and quantum dots. A method of detecting the presence of a disease or condition in a subject by the lipid-polymer hybrid nanoparticle biochip is also disclosed in the specification.

### CROSS REFERENCE

This Application claims the benefit of U.S. Provisional Application No. 62/438,063, filed on Dec. 22, 2016 which is incorporated herein by reference in its entirety.

### SEQUENCE LISTING

The sequence listing, created by PatentIn 3.5 on Nov. 15, 2017 is submitted and is hereby incorporated by reference.

### TECHNICAL FIELD OF THE INVENTION

The present invention relates to a lipid-polymer hybrid nanoparticle biochip and its application for detecting the presence of a disease or condition in a subject. In particular, the lipid-polymer hybrid nanoparticle biochip encapsulates labeling moiety comprises molecular beacons (MB), Toehold-initiated molecular beacons (Ti-MB), biomolecules or components for performing catalyzed hairpin DNA circuit (CHDC), and quantum dots.

### BACKGROUND OF THE INVENTION

Extracellular vesicles (EVs) have emerged as important mediators for intercellular communications involved in many pathophysiological conditions, such as cancer progression and metastasis. EVs are membrane-enclosed vesicles of endocytic origin and contain proteins and nucleic acids. They are secreted by almost all types of cells and enter the circulation. Recently, EV-associated messenger RNA (mRNA) and microRNA (miRNA) have attracted much attention as biomarkers for cancer detection.

Capturing EVs from the body fluids and identifying the encapsulated mRNA/miRNA has become a promising approach to achieve non-invasive cancer diagnosis and monitoring of treatment response. The current method for detecting EV-associated RNAs, such as quantitative reverse transcriptase-polymerase chain reaction (qRT-PCR), needs to extract RNAs by breaking up a large number of EVs prior to analysis, which is time-consuming, laborious and expensive. Especially in early-stage cancer, efficient quantification of EV-associated RNAs with low expression levels remains a challenge. Therefore, it is vital to develop an accurate, simple, fast and inexpensive technique identifying EV-associated RNAs for early cancer diagnosis.

Based on the aforementioned description, it is vital to develop an accurate, simple, fast and inexpensive technique identifying EV-associated RNAs for early cancer diagnosis.

### SUMMARY OF THE INVENTION

In one aspect, the present invention provides a lipid-polymer hybrid nanoparticle biochip. The present lipid-polymer hybrid nanoparticle biochip comprises a gold coating substrate with a surface layer on the gold coating and a nanoparticle, wherein the nanoparticle anchors on the surface layer and encapsulates labeling moiety which comprises molecular beacons (MB), Toehold-initiated molecular beacons (Ti-MB), biomolecules or components for performing catalyzed hairpin DNA circuit (CHDC), and quantum dots.

In another aspect, the present invention discloses a method of detecting the presence of a disease or condition in a subject. The method comprises steps of (1) provides a biological sample obtained from a subject comprises body fluids, cells, tissues and organs; (2) contacts the lipid polymer hybrid nanoparticle chip described in the one aspect with the biological sample from the subject; and (3) detects the target intracellular RNA, DNA, proteins or the combinations existed in the biological sample from the subject by excitation level of a label of labeling moiety that occurs through the capture and incorporation of one comprises cells, cell secreted extracellular vesicles, virus, bacteria, and antigen that corresponds to a disease or condition

In accordance with the present invention, the invented lipid-polymer hybrid nanoparticle biochip relates to a signal-amplifiable biochip selectively and sensitively quantifying EV-associated DNA/RNAs for early cancer detection. Furthermore, the invented lipid-polymer hybrid nanoparticles (LPHN) encapsulating molecular beacons or, biomolecules or components for performing catalyzed hairpin DNA circuit (CHDC) tethered on an Au coated chip, generated a fluorescence signal by capturing extracellular vesicles (EVs) in blood, tissue and other body fluids sample, based on intra-vesicular biomarker such as DNA/RNA targets. LPHN features core-shell-corona structure that facilitates the transfer and mixing of the biomolecules or components for performing CHDC with EV-associated DNA/RNAs when forming LPHN-EV nanocomplex. The catalyzed hairpin DNA circuit is triggered upon target DNA/RNA binding and quickly generate amplified signals. Accordingly, the invented lipid-polymer hybrid nanoparticle biochip is also a signal-amplifiable biochip applies to capture and identify viruses and other pathogens.

In one representative embodiment, the invented lipid-polymer hybrid nanoparticle biochip applies in a method for accurate, simple, fast and inexpensive detecting, identifying, and/or quantifying target EV-associated DNA/RNA in blood or other body fluid sample. The method comprises the following steps: (1) provide a LPHN containing a MB or biomolecules or components for performing catalyzed hairpin DNA circuit (LPHN-MB/CHDC). (2) Tether the LPHN-MB/CHDC on an Au coated glass cover slide. (3) Load the target solution on the LPHN-MB/CHDC tethered chip to form LPHN-EV nanocomplex and (4) generate fluorescence signal measured by total internal reflection fluorescence (TIRF) microscopy. Furthermore, detecting, identifying, and/or quantifying the target EV-associated DNA/RNA by analyzing the fluorescence signal depend on where the fluorescence signal increases in proportion to the concentration of the target EV-associated DNA/RNA.

In the aforementioned method, a developed catalyzed hairpin DNA circuit (CHDC) is utilized for imaging and quantifying low expression target RNAs in EVs. The biomolecules or components for performing CHDC comprise two hairpin DNAs, herein named as H1 and H2. The two hairpin DNAs are allosteric transformations to each other and catalytically triggered by hybridizing with target RNAs. A reporter that is a DNA duplex labeled with a fluorophore and quencher also involve in the CHDC. The CHDC generates multiple signal outputs when hybridized with one single target RNA, compared to conventional molecular beacon (MB) with target RNA in an equivalent reaction ratio (1:1), achieving the goal of signal amplification for effective quantification of RNAs with low copy numbers.

### BRIEF DESCRIPTION OF THE DRAWING

FIG. 1A-1F illustrates the principle and characterization of the invented lipid-polymer hybrid nanoparticles containing catalyzed hairpin DNA circuit (LPHN-CHDC1) biochip. FIG. 1A show stepwise operation of LPHN-TIRF assay, which is composed of link neutravidin (i), tether lipid-polymer hybrid nanoparticle (LPHN) (ii), load extracellular vesicle (EV) (iii) and TIRF measurement (iv); FIG. 1B is schematic illustration of the catalyzed hairpin DNA circuit (CHDC1) consisting of H1, H2 and Reporter for signal amplification of target RNA in LPHN-EV complex (right). One target RNA catalyzes the hybridization of H1 and H2 through toehold-mediated strand displacement reactions for multiple cycles, which further destabilizes Reporter moiety and generates amplified fluorescence (left); FIG. 1C is demonstration of catalysis. Different molar ratios of H1 to H2 were introduced into CHDC1 at t=0. H1=Reporter=80 pmol. 0.0 is the background fluorescence of the absence of H2 and 1.0 is the fluorescence of H1:H2=1:6 at t=120 min. The control traces (black and yellow) show the reaction with no H2 and no target GPC1 DNA, respectively. d-f; FIG. 1D is schematic drawings and transmission electron microscopy (TEM) images of LPHN; FIG. 1E is schematic drawings and transmission electron microscopy (TEM) images of pancreatic cancer-derived EV; and FIG. 1F is schematic drawings and transmission electron microscopy (TEM) images of LPHN-EV complex.

FIG. 2A-2F illustrates Artificial EV (aEV) works as a standard. FIG. 2A is transmission electron microscopy (TEM) micrograph of aEV; FIG. 2B shows calibration curves for fluorescence intensity of GPC1 ssDNA oligo (GPC1-DNA) expression in aEVs using lipoplex nanoparticles containing molecular beacon (LN-MB1), lipoplex nanoparticles containing biomolecules performing catalyzed hairpin DNA circuit (LN-CHDC1), lipid-polymer hybrid nanoparticles containing molecular beacon (LPHN-MB1), and lipid-polymer hybrid nanoparticles containing biomolecules performing catalyzed hairpin DNA circuit (LPHN-CHDC1) individually vs. aEV concentration (37.5, 75.0, 150, 300, 600 and 1200×10⁶ mL⁻¹) (bottom x-axis), and amount of GPC1-DNA in aEV (6.27-, 12.5-, 25.0-, 50.0-, 100- and 200 pg) (upper x-axis), respectively; FIG. 2C is linear scale comparison of limit of detection (LOD) among LN-MB1, LN-CHDC1, LPHN-MB1 and LPHN-CHDC1; FIG. 2D is representative TIRF images of GPC1-DNA expression in varied extremely low concentrations of aEVs (0.18-, 0.37-, 0.75-, 1.5- and 3.0×10⁶ mL⁻¹) by using LPHN-CHDC1; FIG. 2E is a calibration curve for fluorescence intensity of GPC1-DNA expression in aEVs using LPHN-CHDC1 vs low concentration of aEV (0.18-, 0.37-, 0.75-, 1.5- and 3.0×10⁶ mL⁻¹) (bottom x-axis) or low amount of GPC1-DNA in aEV (0.03-, 0.06-, 0.125-, 0.25- and 0.5 pg) (upper x-axis); and FIG. 2F is standard curve of GPC1-DNA expressed in aEVs as the DNA quantity per reaction tube of RT-PCR from 0.125- to 50 pg by serial dilutions, respectively.

FIG. 3A-3F illustrates measurement of GPC1 and KRAS^{G12D} mRNAs in pancreatic AsPC-1 and HPDE6-C7 cell lines. FIG. 3A is representative live cell image of GPC1 mRNA in AsPC-1 and HPDE6-C7 cell lines using lipoplex nanoparticles containing molecular beacon (LN-MB1), lipoplex nanoparticles containing biomolecules performing catalyzed hairpin DNA circuit (LN-CHDC1), lipid-polymer hybrid nanoparticles containing molecular beacon (LPHN-MB1) and lipid-polymer hybrid nanoparticles containing biomolecules performing catalyzed hairpin DNA circuit (LPHN-CHDC1), respectively (Inside upper left, zoomed phase contrast image of individual cell); FIG. 3B is Fluorescence intensity of AsPC-1 cells (signal) and HPDE6-C7 cells (control) treated with LN-MB1, LN-CHDC1, LPHN-MB1 and LPHN-CHDC1, respectively; FIG. 3C is fluorescence signal amplification capability of LN-CHDC1, LPHN-MB1 and LPHN-CHDC1 relative to LN-MB1 based on cell-associated fluorescence of AsPC-1 cells; FIG. 3D is Signal-to-background ratios of LN-MB1, LN-CHDC1, LPHN-MB1 and LPHN-CHDC1 (signal represents fluorescence intensity of AsPC-1 cell; background represents fluorescence intensity of HPDE6-C7 cell); FIG. 3E is A scale of negative Ct value shown for KRAS^{G12D} expression in AsPC-1 and HPDE6-C7 cells, where a higher number represents higher expression and vice versa; and FIG. 3F is representative live cell image of KRAS^{G12D} in AsPC-1 or HPDE6-C7 cell lines using LPHN-CHDC^{KRAS} (Inside upper left, zoomed phase contrast image of individual cell).

FIG. 4A-4K illustrates measurement of GPC1 mRNA in pancreatic cancer cell-derived extracellular vesicles (EVs) and patient serum EVs. FIG. 4A is TIRF images of GPC1 mRNA expression in AsPC-1 EVs (upper row, signal) and HPDE6-C7 EVs (bottom row, control) using lipoplex nanoparticles containing molecular beacon (LN-MB1), lipoplex nanoparticles containing biomolecules performing catalyzed hairpin DNA circuit (LN-CHDC1), lipid-polymer hybrid nanoparticles containing molecular beacon (LPHN-MB1) and lipid-polymer hybrid nanoparticles containing biomolecules performing catalyzed hairpin DNA circuit (LPHN-CHDC1), respectively; FIG. 4B is fluorescence intensity of AsPC-1 EVs (signal) and HPDE6-C7 EVs (control) with the same concentration around 10⁸ mL⁻¹ treated with LN-MB1, LN-CHDC1, LPHN-MB1 and LPHN-CHDC1, respectively; FIG. 4C is fluorescence signal amplification capability of LN-CHDC1, LPHN-MB1 or LPHN-CHDC1 relative to LN-MB1 based on AsPC-1 EV-associated fluorescence; FIG. 4D is signal to background ratios of LN-MB1, LN-CHDC1, LPHN-MB1 and LPHN-CHDC 1 (signal represents fluorescence intensity of AsPC-1 EVs; background represents fluorescence intensity of HPDE6-C7 EVs); FIG. 4E is TIRF images of AsPC-1 EVs with 10-, 50-, 250- and 1000-fold dilution detected by LPHN-CHDC1. EV concentration for each dilution measured by NanoSight LM10 was ˜107 mL−1, ˜2×10⁶ mL⁻¹, ˜4×10⁵ mL⁻¹ and ˜10⁵ mL⁻¹, respectively; FIG. 4F is calibration curve for fluorescence intensity of GPC1 mRNA expression in AsPC-1 EVs using LPHN-CHDC1 vs low concentration of EV. The LOD of AsPC-1 EVs with LPHN-CHDC1 was 57550 per mL; FIG. 4G is representative TIRF images of GPC1 mRNA expression in serum EVs of discovery cohort, healthy donors (n=60), benign pancreatic disease (BPD) patients (n=15), stage I-II pancreatic cancer patients (n=86) and stage III-IV pancreatic cancer patients (n=32), total n=193, using LPHN-CHDC 1 (upper). Fluorescence intensities of GPC1 mRNA expression calculated by METLAB in the discovery cohort (bottom) (Student's t-test, ****P<0.0001); FIG. 4H is cycle threshold value (Ct) for GPC1 mRNA in serum EVs of discovery cohort; FIG. 4I is receiver operating characteristic (ROC) curve analysis of discovery cohort; FIG. 4J is representative TIRF images of GPC1 mRNA expression in serum EVs of validation cohort, healthy donors (n=15), BPD patients (n=8), stage I-II pancreatic cancer patients (n=25) and stage III-IV pancreatic cancer patients (n=23), total n=71, using LPHN-CHDC1 (upper). Fluorescence intensities of GPC1 mRNA expression calculated by METLAB in the validation cohort (bottom) (Student's t-test, ****P<0.0001); and FIG. 4K is ROC curve analysis of validation cohort; and

FIG. 5A-5D illustrates the difference between the molecular beacon and toehold-initiated MB. FIG. 5A shows structure of the conventional MB (Co-MB) and the toehold-initiated MB (Ti-MB). FIG. 5B shows TIRF images of timeline comparison of Co-MB and Ti-MB; FIG. 5C is linear scale comparison of Co-MB and Ti-MB; and FIG. 5D shows stability and signal recovery test of Ti-MB the dissolution curve of the claimed pharmaceutical composition with 0 wt. %, 9 wt. %, 10 wt. % and 11 wt. % coating polymer, respectively. The dissolution method is USP apparatus 1, basket, 100 rpm in 900 mL purified water.

### BRIEF DESCRIPTION OF THE SEQUENCE LISTING

In the following sequence listing, the symbol of [A], [C], [G], and [T] represents a modified base of locked nucleic acid (LNA) of A, C, G and T, respectively.

SEQ ID NO: 1 is a polynucleotide sequence of the molecule beacon 1 (MB1) set forth as follows:

(SEQ ID NO: 1)
CGCGATC[G]CC[T]GC[C]CC[T]GC[T]CA[G]AGGATCGCG

The SEQ ID NO:1 further has a fluorophore at the 5′ end and a quencher at the 3′ end. Preferably, the fluorophore at the 5′ end is FAM and quencher at the 3′ end is BHQ1.

SEQ ID NO: 2 is a polynucleotide sequence of the molecule beacon 2 (MB2) set forth as follows:

(SEQ ID NO: 2)
CGCGATC[G]GA[C]CT[G]AC[C]AG[C]AA[C]CGGATCGCG

The SEQ ID NO:2 further has a fluorophore at the 5′ end and a quencher at the 3′ end. Preferably, the fluorophore at the 5′ end is FAM and quencher at the 3′ end is BHQ1

SEQ ID NO: 3 is a polynucleotide sequence of a first hairpin DNA for performing the first catalyzed DNA circuit (CHDC1-H1) set forth as follows:

(SEQ ID NO: 3)
GCC[T]GCC [C]CT[G]CT [C]AGAG CAATCTCCGCCA
CTCTGAGCAGG ACATCCCA CTTACACC

The CHDC1-H1 is designed for base location at 2,034 of GPC1 mRNA.

SEQ ID NO: 4 is a polynucleotide sequence of a second hairpin DNA for performing the first catalyzed DNA circuit (CHDC1-H2) set forth as follows:

(SEQ ID NO: 4)
CAGAG TGGCGGAGATTG CTCTG AGCAGGCAATCTCCGCCA

The CHDC1-H1 and CHDC1-H2 are allosteric transformations to each other and able to be catalytically triggered by hybridizing with target biomolecules, such as target nucleic acid, RNA and DNA.

SEQ ID NO: 5 is a oligonucleotide sequence of a quencher for performing the first catalyzed DNA circuit (CHDC1-RQ) set forth as follows:

(SEQ ID NO: 5)
ACATCCCA CTTACACC

The SEQ ID NO:5 further has a quencher at the 3′ end. Preferably, the quencher at the 3′ end is BHQ1.

SEQ ID NO: 6 is oligonucleotide sequence of a fluorophore for performing the first catalyzed DNA circuit (CHDC1-RF) set forth as follows:

(SEQ ID NO: 6)
G[G]TG[T]AA[G] TG[G]GA[T]GT CCTGCT

The SEQ ID NO:6 further has a fluorophore at the 5′ end. Preferably, the fluorophore at the 5′ end is FAM.

SEQ ID NO: 7 is a polynucleotide sequence of a first hairpin DNA for performing the second catalyzed DNA circuit (CHDC2-H1) set forth as follows:

(SEQ ID NO: 7)
GGA[C]CTG [A]CC[A]GC [A]ACCG ACCCTCAATCAA
CGGTTGCTGGT AACTTATA CTACCTCC

The CHDC2-H1 is designed for base location at 3,316 of GPC1 mRNA.

SEQ ID NO: 8 is a polynucleotide sequence of a second hairpin DNA for performing the second catalyzed DNA circuit (CHDC2-H2) set forth as follows:

(SEQ ID NO: 8)
AACCG TTGATTGAGGGT CGGTT GCTGGT ACCCTCAATCAA

The CHDC2-H1 and CHDC2-H2 are allosteric transformations to each other and able to be catalytically triggered by hybridizing with target biomolecules, such as target nucleic acid, RNA and DNA.

SEQ ID NO: 9 is a oligonucleotide sequence of a quencher for performing the second catalyzed DNA circuit (CHDC2-RQ) set forth as follows:

(SEQ ID NO: 9)
AACTTATA CTACCTCC

The SEQ ID NO:9 further has a quencher at the 3′ end. Preferably, the quencher at the 3′ end is BHQ1.

SEQ ID NO: 10 is a oligonucleotide sequence of a fluorophore for performing the second catalyzed DNA circuit (CHDC2-RF) set forth as follows:

(SEQ ID NO: 10)
G[G]AG[G]TA[G] TA[T]AA[G]TT ACCAGC

The SEQ ID NO:10 further has a fluorophore at the 5′ end. Preferably, the fluorophore at the 5′ end is FAM.

The aforementioned sequences are summed in TABLE 1

TABLE 1

| | SEQ | | |
|---|---|---|---|
| NAME | ID NO | SEQUENCE | COMMENT |
| MB1 | 1 | CGCGATC[G]CC[T] | FAM at the |
| | | GC[C]CC[T]GC[T] | 5′ end |
| | | CA[G]AGGATCGCG | BHQ1 at the |
| | | | 3′ end |
| MB2 | 2 | CGCGATC[G]GA[C] | FAM at the |
| | | CT[G]AC[C]AG[C] | 5′ end |
| | | AA[C]CGGATCGCG | BHQ1 at the |
| | | | 3′ end |
| CHDC1-H1 | 3 | GCC[T]GCC [C]CT | |
| | | [G]CT [C]AGAG C | |
| | | AATCTCCGCCACTCT | |
| | | GAGCAGG ACATCCC | |
| | | A CTTACACC | |
| CHDC1-H2 | 4 | CAGAG TGGCGGAGA | |
| | | TTG CTCTG AGCAG | |
| | | GCAATCTCCGCCA | |
| CHDC1-RQ | 5 | ACATCCCA CTTACA | BHQ1 at the |
| | | CC | 3′ end |
| CHDC1-RF | 6 | G[G]TG[T]AA[G] | FAM at the |
| | | TG[G]GA[T]GT CC | 5′ end |
| | | TGCT | |
| CHDC2-H1 | 7 | GGA[C]CTG [A]CC | |
| | | [A]GC [A]ACCG A | |
| | | CCCTCAATCAA CGG | |
| | | TTGCTGGT AACTTA | |
| | | TA CTACCTCC | |
| CHDC2-H2 | 8 | AACCG TTGATTGAG | |
| | | GGT CGGTT GCTGG | |
| | | T ACCCTCAATCAA | |
| CHDC2-RQ | 9 | AACTTATA CTACCT | BHQ1 at the |
| | | CC | 3′ end |
| CHDC2-RF | 10 | G[G]AG[G]TA[G] | FAM at the |
| | | TA[T]AA[G]TT AC | 5′ end |
| | | CAGC | |

### DETAILED DESCRIPTION OF THE INVENTION

### Definitions

As used herein, the terms “antibody” and “antibodies” can include, but are not limited to, monoclonal antibodies, polyclonal/multispecific antibodies, human antibodies, humanized antibodies, camelised antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (e.g., anti-Id antibodies to antibodies of the disclosure), and epitope-binding fragments of any of the above. In particular, antibodies include immunoglobulin molecules and immunologically active fragments of immunoglobulin molecules (e.g., molecules that contain an antigen binding site). Immunoglobulin molecules can be of any type (e.g., IgG, IgE, IgM, IgD, IgA and IgY), class (e.g., IgG1, IgG2, IgG3, IgG4, IgA1 and IgA2), or subclass. The antibodies may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes. The antibodies may be monospecific, bispecific, trispecific, or of greater multispecificity.

As used herein, the term “nucleic acid” is term that generally refer to a string of at least two base-sugar phosphate combinations. As used herein, the term includes deoxyribonucleic acid (DNA) and ribonucleic acid (RNA) and generally refer to any polyribonucleotide or polydeoxyribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), miRNA (microRNA), anti-sense RNA, RNAi (RNA interference construct), siRNA (short interfering RNA) or ribozymes. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” and “oligonucleotide” also encompasses a nucleic acid and polynucleotide as defined above. The term “DNA molecule” includes nucleic acids/polynucleotides that are made of DNA.

As used herein, the term “locked nucleic acid (LNA) or LNA nucleosides” are a class of nucleic acid analogues in which the ribose ring is “locked” by a methylene bridge connecting the 2′-O atom and the 4′-C atom in a conformation for Watson-Crick binding, which makes the pairing with a complementary nucleotide strand more rapid and more stable. The LNA has a general chemical structure described as formula (I), where the Base comprises A, G, C and T.

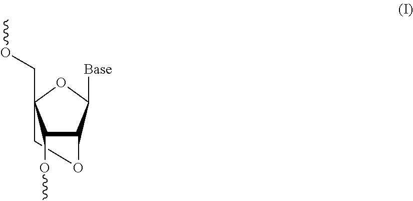

### Embodiments

In one embodiment, the present invention discloses a lipid-polymer hybrid nanoparticle biochip. The lipid-polymer hybrid nanoparticle biochip comprises a gold coating substrate with a surface layer on the gold coating and a nanoparticle, wherein the nanoparticle anchors on the surface layer and encapsulates labeling moiety, which comprises molecular beacons (MB), Toehold-initiated molecular beacons (Ti-MB), biomolecules or components for performing catalyzed hairpin DNA circuit (CHDC), and quantum dots.

In a certain embodiment, the lipid-polymer hybrid nanoparticle biochip has the surface layer being self-assembly monolayer selected from the group consisting of 2-mercaptoethanol (βME), 6-mercaptohexanol, Biotin-PEG-thiol (HS-PEG-Biotin), thiol-backfiller molecules and combinations thereof.

In certain embodiment, surface of the nanoparticle further functionalizes with one comprises avidin-biotin, fluorescein-anti-FITC, hapten linkages of antibody molecules, peptides, carbohydrate, DNA and RNA.

In certain embodiment, the nanoparticle is formed by a polymer and a lipid. Typically, the polymer comprises Polyethylenimine (PEI), Poly-L-Lysine (PLL), Poly-D-Lysine (PDL), Poly (ε-caprolactone) (PCL), Polylactide (PLA) and Poly (Lacitide-co-Glycolide) (PLGA). The lipid comprises 1,2-di-O-octadecenyl-3-trimethylammonium propane (DOTMA), 1,2-dioleoyl-3-trimethylammonium-propane (DOTAP), 3β-[N-(N′,N′-dimethylaminoethane)-carbamoyl]cholesterol hydrochloride (DC-Cholesterol), and other ionizable lipids which include 1,2-di-O-octadecenyl-3-dimethylammonium propane (DODMA), 1,2-dioleoyl-3-dimethylammonium-propane (DODAP). Other non-ionizable lipids include L-α-phosphatidylcholine (EggPC, SoyPC), Cholesterol, 1,2-dipalmitoyl-sn-glycero-3-phosphocholine (DPPC), and other saturated fatty acid, 1,2-dioleoyl-sn-glycero-3-phosphocholine (DOPC), and other helper lipids and 1,2-distearoyl-sn-glycero-3-phosphoethanolamine-N-[methoxy(polyethylene glycol)] (DSPE-PEG), and PEG phospholipids.

In certain embodiment, the molecular beacons comprise a oligonucleotide, wherein the oligonucleotide has a fluorophore at 5′ end and a quencher at 3′ end. Preferably, the oligonucleotide is selected from the group consisting of SEQ ID NO: 1 and SEQ ID NO: 2.

In certain embodiment, the fluorophore at 5′ end comprises FAM, TET, HEX, Cyanine dyes (Cy2, Cy3, Cy3B, Cy3.5, Cy5, Cy5.5, Cy7), TMR, ROX, JOE, Texas red, LC red 640, and LC red 705.

FAM represents Carboxyfluorescein.

TET represents carboxy-2′,4,7,7′-tetrachlorofluorescein succinimidyl ester.

HEX represents carboxy-2,4,4,5,7,7-hexachlorofluorescein succinimidyl ester.

Cyanine dyes as the fluorophore include Cy2, Cy3B, Cy3.5, Cy5, Cy5.5, Cy7.

TMR represents Tetramethylrhodamine.

ROX represents carboxy-X-rhodamine.

JOE represents carboxy-4′,5′-dichloro-2′,7′-dimethoxyfluorescein.

In certain embodiment, the quencher at 3′ end comprises Black Hole Quenchers (BHQ-0, BHQ-1, BHQ-2, BHQ-3, Deep Dark Quenchers (DDQ-I, DDQ-II), QSY-7, QSY-21, Dabcyl, Eclipse, Iowa Black FQ, and Iowa Black RQ.

QSY-7 represents QSY7 carboxylic acid, succinimidyl ester.

QSY-21 represents QSY 21 carboxylic acid, succinimidyl ester.

In another embodiment, the Ti-MB comprises a oligonucleotide, wherein the oligonucleotide has a stem of 5 to 50 base pairs and a loop of 1 to 100 bases, a toehold domain of 1 to 50 complementary bases to target DNA, RNA or the combination is added at the end of the stem. wherein the oligonucleotide has a fluorophore at 5′ end and a quencher at 3′ end.

In certain embodiment, the biomolecules or components for performing catalyzed hairpin DNA circuit (CHDC) comprises a first hairpin DNA, a second hairpin DNA, a fluorophore-labeled oligonucleotide strand (RF) has 5 to 100 bases and a quencher-labeled oligonucleotide strand (RQ) has 5 to 100 bases complementary to the RF. The first hairpin DNA comprises a stem of 5 to 50 base pairs and a loop of 1 to 100 bases, a toehold domain of 1 to 50 complementary bases to a target DNA/RNA is added at the end of the stem and the second hairpin DNA comprises a stem of 5 to 50 base pairs and a loop of 1 to 100 bases, a toehold domain of 1 to 50 complementary bases to domain of the first hairpin DNA, and the first hairpin DNA and the second hairpin DNA form a duplex.

In one embodiment, the first hairpin DNA is SEQ ID NO: 3, the second hairpin DNA is SEQ ID NO: 4, the quencher-labeled oligonucleotide strand (RQ) is SEQ ID NO: 5 has BHQ1 at the 3′ end and the fluorophore-labeled oligonucleotide strand (RF) is SEQ ID NO: 6 has FAM at the 5′ end.

In another embodiment, the first hairpin DNA is SEQ ID NO: 7, the second hairpin DNA is SEQ ID NO: 8, the quencher-labeled oligonucleotide strand (RQ) is SEQ ID NO: 9 has BHQ1 at the 3′ end and the fluorophore-labeled oligonucleotide strand (RF) is SEQ ID NO:10 has FAM at the 5′ end.

In a certain embodiment, the lipid-polymer hybrid nanoparticle biochip is applied to capture extracellular vesicle (EV), virus or cell for detecting one comprises DNA, RNA and protein.

In another embodiment, the present invention discloses a method of detecting the presence of a disease or condition in a subject. The method comprises following steps. (1) Provide a biological sample obtained from a subject comprises body fluids, cells, tissues and organs. (2) Contact the lipid polymer hybrid nanoparticle chip disclosed in the aforementioned embodiment with the biological sample from the subject. And (3) detect the target intracellular RNA, DNA, proteins or the combinations existed in the biological sample from the subject by excitation level of a label of labeling moiety that occurs through the capture and incorporation of one comprises cells, cell secreted extracellular vesicles, virus, bacteria, and antigen that corresponds to a disease or condition.

In one embodiment, the labeling moiety comprises molecular beacon, toehold initiated molecular beacon (Ti-MB), biomolecules or components for performing catalyzed hairpin DNA circuit (CHDC) and quantum dot.

In one embodiment, the molecular beacon is oligonucleotide selected from the group consisting of SEQ ID NO: 1 and SEQ ID NO: 2, and the oligonucleotide has a fluorophore at the 5′ end and a quencher at the 3′ end.

In certain embodiment, the biomolecules or components for performing catalyzed hairpin DNA circuit (CHDC) are selected from the group consisting of a first polynucleotide composition and a second polynucleotide composition. The first polynucleotide composition comprises SEQ ID NO: 3, SEQ ID NO: 4, SEQ ID NO: 5 has BHQ1 at the 3′ end and SEQ ID NO: 6 has FAM at the 5′ end. The second polynucleotide composition comprises SEQ ID NO:7, SEQ ID NO: 8, SEQ ID NO: 9 has BHQ1 at the 3′ end and SEQ ID NO:10 has FAM at the 5′ end.

In one embodiment, the biological sample from the subject comprises blood serum, blood plasma, whole blood, nasal aspirates, saliva, urine, sputum, feces, cell lysate, dialysis sampling, tissue biopsy, cell media, and a combination thereof.

In one embodiment, the disease or condition is selected from the group consisting of: cancer, viral infection, bacterial infection, heart attack, stroke, rhabdomyolysis, fertility, diabetes, obesity, metabolic syndrome, sepsis, inflammatory response, food safety, tuberculosis, and a combination thereof.

In one embodiment, the cancer comprises lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colon-rectal cancers, prostatic cancer, pancreatic cancer, and cancer cachexia.

To disclose and interpret the present invention in details, the following embodiments are also disclosure.

In one embodiment of the present invention, the cationic lipid polymer hybrid nanoparticle (LPHN) biochip could selectively and sensitively detect the target intracellular DNA/RNA by the excitation of a label of the labeling moiety. The excitation of a label of the labeling moiety occurs through the capture and incorporation of cell, cell secreted extracellular vesicles, virus, bacteria, or antigen that corresponds to a particular disease or condition into the LPHN.

In one embodiment of the present invention, the method of diagnosing the presence of a disease or condition comprising obtaining a biological sample from a subject comprises the following steps, Contact a LPHN biochip with the biological sample from the subject, wherein the LPHN comprises one or more labeling moieties. The labeling moieties include molecular beacon, toehold-initiated molecular beacon, biomolecules or components for performing catalyzed hairpin DNA circuit (CHDC) and quantum dots. The aforementioned labeling moieties encapsulated in the LPHN. Moreover, the LPHN further comprises one or more surface targeting moieties. The surface targeting moieties comprise chemicals with positive charge, antibodies, peptides, carbohydrates, DNA/RNA and their mixtures. The surface targeting moieties on the LPHN surface functionalize as receptors for binding specific target cells, cell secreted extracellular vesicles, virus, bacteria, or antigen that corresponds to a particular disease or condition. As a result, the presence of a disease or condition is indicated by the excitation of a label of the labeling moiety that occurs through the capture and incorporation of a cell, cell secreted extracellular vesicle, virus, bacteria, or antigen that corresponds to a particular disease or condition into the LPHN.

In one embodiment of the present invention, the antigen can be a viral antigen or virus, bacterial antigen or bacteria, toxin, cancer related antigen such as a cancer cell, cancer cell secreted extracellular vesicle, or cancer protein.

In one embodiment of the present invention, the LPHN biochip composes a gold coating of a solid substrate with the substrate being glass, silicon wafer, polymer, ceramics or any solid materials, where the surface immobilized self-assembled monolayer (SAM) comprises 2-mercaptoethanol (BME), Biotin-PEG-SH, 6-Mercaptohexanol, 16-mercaptohexadecanoic acid (MHA), and other thiol-backfiller molecules.

In one embodiment of the present invention, the LPHN biochip comprises but not limited to a polymer mixture and a lipid mixture. The polymer mixture comprises Polyethylenimine (PEI), Poly-L-Lysine (PLL), Poly-D-Lysine (PDL), Poly (ε-caprolactone) (PCL), Polylactide (PLA) and, Poly (Lacitide-co-Glycolide) (PLGA). The lipid mixture comprises 1,2-di-O-octadecenyl-3-trimethylammonium propane (DOTMA), 1,2-dioleoyl-3-trimethylammonium-propane (DOTAP), 3β-[N-(N′,N′-dimethylaminoethane)-carbamoyl]cholesterol hydrochloride (DC-Cholesterol), and other ionizable lipids, such as 1,2-di-O-octadecenyl-3-dimethylammonium propane (DODMA), 1,2-dioleoyl-3-dimethylammonium-propane (DODAP), and other non-ionizable lipids, such as L-α-phosphatidylcholine (EggPC, SoyPC), Cholesterol, 1,2-dipalmitoyl-sn-glycero-3-phosphocholine (DPPC). Additionally, fatty acids, 1,2-dioleoyl-sn-glycero-3-phosphocholine (DOPC), other helper lipids such as 1,2-distearoyl-sn-glycero-3-phosphoethanolamine-N-[methoxy(polyethylene glycol)] (DSPE-PEG), and other PEG phospholipids are also included in the embodiment.

In one embodiment, the LPHN layer is formed by covalent or semi-covalent attachment of the LPHN to the gold coated substrate with self-assembled monolayer through avidin-biotin, digoxigenin (Dig)-anti-Dig, fluorescein-anti-FITC or other hapten linkages.

Embodiments of the present invention include a LPHN biochip where the LPHN comprises at least one labeling moiety, for example, a molecular beacon, toehold initiated molecular beacon (Ti-MB), biomolecules or components for performing catalyzed hairpin DNA circuit (CHDC), or quantum dot. In an embodiment, the labeling moiety can include a radio label, enzyme-linked detection systems, antibody-mediated label detection, fluorescent labels and dyes, fluorescent change probes and primers, such as molecular beacons, Amplifluors, FRET probes, hairpin quenched probes, cleavable FRET probes, TaqMan probes, scorpion primers, fluorescent triplex oligos, fluorescent water-soluble conjugated polymers, PNA probes and QPNA probes.

Embodiments of the present invention include a LPHN biochip where the Ti-MB comprises a stem of 5 to 50 base pairs and a loop of 1 to 100 bases, a toehold domain of 1 to 50 complementary bases to target DNA/RNA is added at the end of the stem.

Embodiments of the present invention include a LPHN biochip where catalyzed hairpin DNA circuit (CHDC) comprises two hairpin DNAs whose allosteric transformations can be catalytically triggered by hybridizing with target nucleic acid, and at least one fluorophore-labeled oligonucleotide strand (RF), and at least one quencher-labeled oligonucleotide strand (RQ). In an embodiment, a first hairpin DNA (H1) comprises a stem of 5 to 50 base pairs and a loop of 1 to 100 bases, a toehold domain of 1 to 50 complementary bases to target DNA/RNA is added at the end of the stem. A second hairpin DNA (H2) comprises a stem of 5 to 50 base pairs and a loop of 1 to 100 bases, a toehold domain of 1 to 50 complementary bases to domain of the H1. RF has 5 to 100 bases. RQ has 5 to 100 bases complementary to RF.

In one embodiment of the present invention, a LPHN biochip for capturing target cells, extracellular vesicles, virus or bacteria with the contained mRNAs, microRNAs and/or proteins is provide. The target cells, extracellular vesicles, virus or bacteria with the contained mRNAs, microRNAs and/or proteins are detected by molecular probes which comprises molecular beacons, Ti-MB, biomolecules or components for performing CHDC, quantum dots and/or other sensing molecules and particles using a total internal reflective fluorescence (TIRF) microscope, fluorescence microscope, plate reader, portable fluorescence detector or flow cytometry.

Embodiments of the present invention include an LPHN biochip where the disease or condition is selected from the group consisting of: cancer, viral infection, bacterial infection, heart attack, stroke, rhabdomyolysis, fertility, diabetes, obesity, metabolic syndrome, sepsis, inflammatory response, food safety, tuberculosis, and a combination thereof. In an embodiment, the LPHN biochip is used to detect and/or treat any disease and/or condition diagnosed by an nucleic acid, protein or peptide.

Embodiments of the present invention include an LPHN biochip. The disease or condition is a cancer and the antigen or EV detected by the LPHN biochip is derived from a cancer which is selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colon-rectal cancers, prostatic cancer, pancreatic cancer, or cancer cachexia.

In an embodiment of the present invention, the biological sample comprises blood serum, whole blood, nasal aspirates, saliva, urine, feces, cell lysate, dialysis sampling, tissue biopsy, cell media, and a combination thereof. In another embodiment, the biological sample is unprocessed. For example, whole blood, saliva, or urine samples that have not been processed through dilution or purification steps. In another embodiment, the method is used in a basic research laboratory to detect, quantify, or identify cells, EVs, nucleic acids, proteins, or peptides. In another embodiment, the method is used in a clinical laboratory to detect, quantify, and/or identify biomarkers of disease. In yet another embodiment, the method is used at the point-of-care (POC) to detect, quantify, and/or identify biomarkers of disease.

The disclosed method utilizes biological sample obtain from a subject. As used herein “subject” can refer to any human, non-human primate, dog, cat, cow, horse, pig, rat, mouse, gerbil, guinea pig, or fish. A subject can include but is not limited to a patient having previously been diagnosed with a general condition but in need of specific diagnosis.

### EXAMPLES

### General Principles

The present disclosure includes examples of a novel yet simple to implement non-invasive nanotechnology-enabled approach for early cancer detection using a nanoparticle-based biochip. Such biochip can capture and quantify EV-associated RNAs in a rapid, highly effective and non-isolative manner. Biomolecules or components for performing non-enzymatic catalyzed hairpin DNA circuit (CHDC) encapsulated in lipid-polymer hybrid nanoparticle (LPHN) generated amplified fluorescence signals upon one target RNA binding within the LPHN-EV nanocomplex, as demonstrated with newly developed artificial EVs and cancer cell-derived EVs. This approach applied to recognize cancer cells and mutant cells as well. LPHN-CHDC biochip with signal-amplification capability selectively and sensitively identified low-expression glypican-1 mRNA in serum EVs, successfully distinguishing healthy donors and patients with a benign pancreatic disease from patients with early- and late-stage pancreatic cancer. This simple to implement biochip approach, which elicits and propagates powerfully trustworthy fluorescence signals through a 2-h exoteric treatment with only 10 μL patient serum, could find a wide variety of applications in the development of novel non-invasive extracellular vesicles or viruses-based early diagnostic and screening tools.

Extracellular vesicles have emerged as important mediators for intercellular communications involved in many pathophysiological conditions, such as cancer progression and metastasis. EVs are membrane-enclosed vesicles of endocytic origin and contain proteins and nucleic acids. They are secreted by almost all types of cells and enter the circulation. More recently, EV-associated messenger RNA (mRNA) and microRNA (miRNA) have attracted much attention as biomarkers for cancer detection. Capturing EVs from the body fluids and identifying the encapsulated mRNA/miRNA has become a promising approach to achieve non-invasive cancer diagnosis and monitoring of treatment response. The current method for detecting EV-associated RNAs, such as quantitative reverse transcriptase-polymerase chain reaction (qRT-PCR), needs to extract RNAs from large-volume condensed EVs prior to analysis, which is time-consuming, laborious and expensive. Especially in early-stage cancer, efficient quantification of EV-associated RNAs with low expression levels remains a challenge. Therefore, it is vital to develop an accurate, simple, fast and inexpensive technique identifying EV-associated RNAs for early cancer diagnosis.

The present examples disclose a developed catalyzed hairpin DNA circuit (CHDC) for imaging and quantifying low expression target RNAs in EVs. The biomolecules or components for performing CHDC consists of two hairpin DNAs whose allosteric transformations can be catalytically triggered by hybridizing with target RNAs, and a reporter which is a DNA duplex labeled with a fluorophore and quencher. The CHDC could generate multiple signal outputs when hybridized with one single target RNA (m:1), compared to conventional molecular beacon (MB) with target RNA in an equivalent reaction ratio (1:1), achieving the goal of signal amplification for effective quantification of RNAs with low copy numbers. With complementary characteristics of both lipoplex nanoparticle (LN) and polymeric nanoparticle, cationic lipid-polymer hybrid nanoparticle (LPHN) has emerged as an effective nanocarrier for genes delivery owing to its superior biocompatibility, structural stability and encapsulation efficiency (EE). However, to the best of our knowledge, there has been no report performing CHDC inside LPHN to quantify EV-associated RNAs for high signal gain. Here, we develop a smart system termed signal-amplifiable LPHN-CHDC biochip capable of highly selective and sensitive quantification of target RNAs in EVs to achieve non-invasive early cancer diagnosis. Glypican-1 (GPC1) is a membrane-anchored protein that has recently been identified in breast and pancreatic cancer EVs, while non-cancer EVs did not exhibit GPC1 protein. Thus, we selected GPC1 mRNA as a model biomarker, which is supposed to be enriched in cancer pancreatic cancer cell secreted EVs rather than EVs secreted from normal cells, to verify our novel assay compared to the widely used qRT-PCR for signal amplification capacity and, further, challenged to detect early pancreatic cancer. Our findings indicate LPHN-CHDC biochip as a resourceful yet simple to implement signal-amplification tool for precise early cancer detection.

The following reagents and materials used in the examples comprise 1,2-Di-O-octadecenyl-3-trimethylammonium propane (chloride salt) (DOTMA), 1,2-distearoyl-sn-glycero-3-phosphoethanolamine-N-[biotinyl (polyethylene glycol)-2000] (ammonium salt) (Biotin-PEG-DSPE), 1,2-dioleoyl-sn-glycero-3-phosphoethanolamine (DOPE) and 1,2-Dimyristoyl-sn-glycerol methoxypolyethylene glycol (DMG-PEG) were supplied by Avanti Polar Lipids, Inc. Poly (D,L-lactide-co-glycolide) (PLGA) (lactide:glycolide 75:25, ester-terminated, Mw 4,000-15,000), Cholesterol, linoleic acid (LA), β-mercaptoethol (βME) and 3-mercaptopropyl-trimethoxysilane (MPTMS) were purchased from Sigma-Aldrich (St. Louis, Mo.). Biotin-PEG-SH was supplied by Nanocs Inc. Oligonucleotides were obtained from Sigma-Aldrich, with purity and yield confirmed by mass spectrometry and HPLC, respectively. Ultrapure water (EMD Millipore) was used throughout the experiment. All other reagents and solvents were of analytic grade.

### Design of MBs and Biomolecules or Components for Performing Catalyzed Hairpin DNA Circuit (CHDC)

The MBs used in the following examples for targeting GPC1 mRNA comprise MB1 and MB2. The MB1 is SEQ ID NO:1 has FAM at the 5′ end and BHQ1 at the 3′ end. The MB2 is SEQ ID NO:2 has FAM at the 5′ end and BHQ1 at the 3′ end. Both MB1 and MB2 were designed based on NCBI reference sequence of GPC1 (NM_002081.2). The MB1 and MB2 were complementary for two different locations of GPC1 mRNA (base location: 2,034 and 3,316).

Preferably, the biomolecules or components for performing catalyzed hairpin DNA circuit (CHDC) are combined or formed to a polynucleotide composition, which comprise 4 numbers of polynucleotides at least. A polynucleotide composition designed for base location 2,034 of GPC1 mRNA comprises SEQ ID NO:3, SEQ ID NO:4, SEQ ID NO:5 has BHQ1 at the 3′ end. and SEQ ID NO:6 has FAM at the 5′ end. The another polynucleotide composition designed for base location 3,316 of GPC1 mRNA comprises SEQ ID NO:7, SEQ ID NO:8, SEQ ID NO:9 has BHQ1 at the 3′ end and SEQ ID NO:10 has FAM at the 5′ end.

To improve the thermal stability and nuclease resistance of MBs and the biomolecules or components for performing CHDCs for long-term imaging of mRNA at 37° C., Some bases in the sequence of the polynucleotides are modified. Typically, locked nucleic acid (LNA) nucleotides were incorporated into polynucleotide strands. In this way, the invented polynucleotides can efficiently target specific mRNA without interference from cellular nucleases and proteins. LNA nucleosides are a class of nucleic acid analogues in which the ribose ring is “locked” by a methylene bridge connecting the 2′-O atom and the 4′-C atom in the ideal conformation for Watson-Crick binding, which makes the pairing with a complementary nucleotide strand more rapid and more stable.

### Optimization of Polynucleotides for Performing CHDC

10 μM stock of Reporter (RF:RQ) was prepared by annealing 10 μM RF and 20 μM RQ. Excess RQ ensures efficient quenching of RF but does not interfere with the readout of H1:H2. H1 and H2 were individually refolded by heating to 90° C. for 2 min followed by slowly decreasing the temperature to 4° C. at a rate of 0.1° C. s⁻¹. All reagents were prepared in 1×DPBS buffer (Gibco BRL). All kinetic measurements were carried out at 37° C. The reactions were started by the addition of different molar ratios of H1:H2 with 1 nM target GPC1 DNA at H1=Reporter=80 pmol. Reaction mixtures (50 μL for each aliquot) were added into different wells of a 96-well plate. Fluorescence signal was measured by TECAN Sunrise plate reader with temperature control at each 10-min time point.

### Preparation of LPHN-MB1 and LPHN-CHDC1

MB1 or CHDC1-encapsulated LPHNs were prepared by a w₁/o/w₂ solvent evaporation method with some modifications. 50 μL aqueous solution (w₁) of MB1 (3.2 μM) or CHDC1 (molar ratio of H1:H2:Reporter=1:6:1) for GPC1 mRNA was emulsified in 250 μL organic solvent (o) containing PLGA (3 mg) by ultrasonic (Branson Digital Sonifier, USA) for 15 s. DOTMA and Biotin-PEG-DSPE (92:8 weight ratio) were dissolved in 4% ethanol aqueous solution, which was preheated at 65° C. for 15 min. Thereafter, the primary emulsion (w₁/o) was poured into 600 μL DOTMA/Biotin-PEG-DSPE solution (w₂) followed by two steps of re-emulsification by ultrasonic. The double emulsion (w₁/o/w₂) was subsequently dispersed into 1.1 mL DOTMA/Biotin-PEG-DSPE solution (w₂) and then vacuumed to completely remove the solvents. The formed LPHN-MB1 or LPHN-CHDC 1 suspensions were incubated at 4° C. and used immediately.

### Preparation of LN-MB1 and LN-CHDC1

25 μL MB1 (6.4 μM) or CHDC1 (molar ratio of H1:H2:Reporter=1:6:1) for GPC1 mRNA in PBS were mixed with 20 μL lipid mixture (DOTMA:Cholesterol:Biotin-PEG-DSPE=52:46:2 molar ratio) in ethanol (10 mg/mL) by ultrasonic for 5 min. Then the oligonucleotides/lipid mixture was injected into 455 μL PBS and further sonicated for 5 min. The formed LN-MB 1 or LN-CHDC 1 suspensions were incubated at 4° C. and used immediately.

### Preparation of Artificial EVs (aEVs)

Briefly, 30 μL mixture of GPC1 DNA with scramble DNA (1:99 molar ratio) in PBS were mixed with 20 μL lipid mixture (DOPE:LA:DMG-PEG=52:46:2 molar ratio) in ethanol by ultrasonic for 5 min, then the mixture was injected into 550 μPBS for another 5 min sonication. The formed aEV suspensions were incubated at 4° C. as the stock solution, which was further diluted by PBS into 1.2%, 2.5%, 5%, 10%, 20% and 40% as work solutions.

### Physicochemical Characterization

For structural characterization, LPHNs, patient serum EVs and aEVs were observed by cryo-transmission electron microscopy (cryo-TEM). To visualize the fusion of LPHNs with patient serum EVs, LPHNs and EVs (1:1 concentration ratio) were incubated at 37° C. for 30 min. To prepare cryo-TEM specimen, a 3-μL drop of sample was placed on a holey carbon-coated copper grid and immediately frozen in liquid ethane cooled with liquid nitrogen. Specimen was then maintained at −170° C. using a Gatan 626-DH cryoholder and viewed in a JEOL-2100 TEM at 200 kV. Images were recorded with a 4k×4k low-dose CCD camera. Biological Atomic Force Microscopy (Bio-AFM) were used to image LPHN-CHDC 1 biochip before and after loading serum EVs in the PBS buffer solution. Bio-AFM (SPA-400, Japan) was mounted on an inverted microscope, TE-2000-U (Nikon, Japan). Au-coated Si₃N₄ pyramidal cantilevers used had a nominal spring constant of 0.09 N/m. (DNP-20, Veeco). Imaging was performed using contact mode in fluid and collected using a scan rate varied from 0.5 to 2 Hz.

Particle sizes and concentrations of LNs, LPHNs and aEVs were analyzed by NanoSight LM10 (NanoSight Ltd., Amesbury, UK) and their zeta potentials were determined by dynamic light scattering using a Zetasizer Nano ZS (Malvern Instrument Ltd., UK) at 25° C. For measurement, samples were diluted to the appropriate concentration with Millipore water.

To determine the encapsulation efficiency, FAM-labeled oligo DNA (F-ODN) were firstly encapsulated in the LNs, LPHNs, and aEVs, respectively. The amount of encapsulated F-ODN in the nanoparticles is calculated by subtracting the amount of F-ODN present in the supernatant after centrifugation from the amount of F-ODN initially added. A standard curve correlating fluorescence and F-ODN concentration was used to determine the amount F-ODN in the supernatant 35. The fluorescence intensity was measured by fluroskan ascent reader (Thermo Labsystems, Finland) using λex=488 nm, λem=520 nm. The EE was calculated from the following equation:

EE(%)=(W₀−Wₜ)/W₀×100%

Where W₀ is the amount of initial F-ODN; Wₜ is the F-ODN amount in the supernatant.

### Biochip Fabrication

A glass cover slip (ThermoFisher Scientific, Waltham, Mass.) was carefully cleaned by using Millipore water and ethanol two times alternatively, and dried under flowing nitrogen. The cleaned surface of glass cover slip was then activated with UV/O₃ using jelight Model 42 UVO cleaner with O₃ capture system (Jelight Company Inc., CA). The activated surface was modified with vapor of MPTMS in low pressure vacuum chamber for 10 min. A thin Au layer (15 nm) was deposited on the glass cover slip over an MPTMS layer as a glue layer using a Denton e-beam evaporator (DV-502A, Moorestown, N.J.). For immobilization, the freshly prepared Au coated glass cover slip was transferred directly to linker solution, a mixture of Biotin-PEG-SH and βME (5:95 molar ratio) in 200 proof ethanol, for 16 h at room temperature in the dark, the excess mixture physically adsorbed on the surface of glass cover slip was removed via ethanol rinse (˜10 s). Following the formation of a self-assembled Biotin-PEG-SH/βME monolayer, a pre-molded 24-well PDMS plate (4 by 6 array, 4 mm well diameter) was attached on the treated surface of glass cover slip. Then, 10 μL neutravidin solution (ThermoFisher Scientific, Waltham, Mass.) was added in each well of the chip and incubated at room temperature for 30 min under shaking (500 rpm) (Titer plate shaker, Lab-line instruments, Inc.). Unbound neutravidin was automatically washed away using PBS buffer solution by MultiFlo FX (BioTek Instruments, Inc.). Thereafter, 10 μL LPHN-CHDC1 suspension was added in the well and tethered on the chip surface by biotin-avidin linkage via incubating at room temperature for 30 min under shaking (500 rpm), and the unbound LPHN-CHDC1 were automatically washed away using PBS buffer solution. For the fabrication of LN-MB1, LN-CHDC 1 or LPHN-MB1 biochip, 10 μL LN-MB1, LN-CHDC for LPHN-MB1, instead of LPHN-CHDC1 suspension was added in the well and tethered on the chip surface.

### Cell Culture and EV Isolation

AsPC-1, which is a human pancreatic carcinoma cell line with overexpressed GPC1 mRNA, was chosen as test cell. HPDE6-C7, which is a normal pancreatic duct epithelial cell line, was chosen as negative control cell. AsPC-1 cells (American Type Culture Collection (ATCC) were maintained in RPMI-1640 Medium (11875-093, ThermoFisher Scientific, Waltham, Mass.) with 10% FBS (fetal bovine serum, Invitrogen, Carlsbad, Calif.). HPDE6-C7 cells (Kerafast, Inc., Boston, Mass.) were maintained in Keratinocyte Serum Free Medium (KSFM) (17005-042, ThermoFisher Scientific, Waltham, Mass.) supplemented with 25 mg/500 mL Bovine Pituitary Extract (BPE) (13028-014, ThermoFisher Scientific, Waltham, Mass.) and 2.5 μg/500 mL Epidermal Growth Factor (EGF) (ThermoFisher Scientific, Waltham, Mass.). All cell lines were grown without antibiotics in an atmosphere of 5% CO₂, 99% relative humidity at 37° C. Cells were plated in T225 cm² flasks and grown to 80-90%) confluence. Next, cell culture medium was collected and centrifuged at 4,000×g for 10 min to remove cells. The supernatant was then centrifuged at 10,000×g for 10 min to remove cell debris. Then, the supernatant was filtered using a 0.22 μm pore filter (syringe filter, 6786-1302, GE Healthcare). The filtered supernatant containing cell secreted EVs was directly used for biochip detection. The filtered supernatant was collected and ultra-centrifuged at 100,000×g for 90 min at 4° C. to retain the precipitated pellets of EVs. The EV pellets were washed with 30 mL PBS once, precipitated by second ultra-centrifugation at 100,000×g for 90 min at 4° C., supernatant was discarded. EVs used for RNA extraction were resuspended in 500 μL of Trizol. EVs used for cryo-TEM were resuspended in 100 μL PBS. 10 μL of this EVs sample used for NanoSight LM10 analysis were diluted in PBS at 1:100 volume ratio.

### EV Isolation from Human Serum Samples

Human serum EVs were isolated using a previously reported protocol with minor alterations. 250 μL cell-free serum samples were thawed on ice. Serum was diluted in 10 mL PBS and filtered through 0.22 μm pore filter, and ultra centrifuged at 150,000×g overnight at 4° C. Afterwards, the EV pellets were washed in 10 mL of PBS, and a second step of ultracentrifugation (150,000×g, 4° C.) was performed for 2 h. The supernatant was discarded. EVs used for RNA extraction were resuspended in 500 μL Trizol. EVs used for cryo-TEM were resuspended in 100 μL PBS. 10 μL of these EV pellets was diluted by PBS at 1:100 volume ratio for NanoSight LM10 analysis.

### RNA Extraction of Cells and EVs

Following the manufacturer's protocol, RNA of cells and EVs was isolated using Trizol Plus RNA purification kit (ThermoFisher Scientific, Waltham, Mass.).

### qRT-PCR Measurement of Target RNA Expression

100 ng of RNA extracted from 2.0×10₈ EVs was reverse-transcribed using SuperScript II RNase-Reverse Transcriptase system (18064-014, ThermoFisher Scientific) following the manufacturer's procedure on a 7300 Sequence Detector System (Applied Biosystems). Primers for GPC1 mRNA (Sigma-Aldrich) at two different locations (2,034; 3,316) were designed as shown in Table 2.

TABLE 2

| | Abbre- | DNA sequence, |
|---|---|---|
| Name | viation | listed 5′ to 3′ |
| Primer 1-forward | P1-F | ATATTTAATTCACCTCAG |
| Primer 1-reverse | P1-R | TCATACAAAATTAAAAGG |
| Primer 2-forward | P2-F | CTGCTTTGCTTTTCATCA |
| Primer 2-reverse | P2-R | AAACATCTAAAGTCAGGTTC |
| Primer′-forward | KRAS-P′-F | ACTTGTGGTAGTTGGAGCAGA |
| (for KRASG12D) | | |
| Primer′-reverse | KRAS-P′-R | TTGGATCATATTCGTCCACAA |
| (for KRASG12D) | | |

Primers for KRAS^{G12D} mRNA (Sigma-Aldrich) used previously designed sequences. Briefly, the mutated base of KRAS^{G12D} was kept at the 3′ end of the forward primer. An additional altered base was included two positions before the KRAS mutation to increase the specificity of the amplification of the mutant KRAS allele. Forward primer sequence for KRAS^{G12D} mRNA: 5′-ACTTGTGGTAGTTGGAGCAGA-3′ (italicized bases represent mutations corresponding to the KRAS mutant). Reverse primer for KRAS^{G12D} mRNA: 5′-TTGGATCATATTCGTCCACAA-3′. PCR was performed in a 20 μL reaction tube containing 2 μL of template DNA, 0.8 μL of each forward and reverse primers (10 pmol), 10 μL 2× PowerUp SYBR Green Master Mix (Applied Biosystems), 6.4 μL of nuclease free water. Amplification was carried out under the following conditions: 95° C. for 2 min, 40 cycles of 95° C. for 15 s, 58° C. for 30 s, 70° C. for 30 s; endless 4° C. RNA expression levels were normalized to the level of spiked cel-miR-39 (Assay ID: 000200, Applied Biosystems).

### TIRF Measurements and Image Analysis

10 μL sample such as aEVs, cells, cell-secreted EVs and patient serum was added in each well of biochip (4 by 6 array, 4 mm well diameter). The biochip was incubated in the dark at 37° C. and 99% relative humidity for 2 h before measurement. Total Internal Reflection Fluorescence (TIRF) Microscopy (Nikon Eclipse Ti Inverted Microscope System) was used to record and analysis sample images. TIRF occurs at the interface between optically dense medium, such as glass and aqueous solution. By adjusting the angle of incidence to a critical point, the excitation beam reflects back into glass and generates evanescent wave which has maximum of intensity at the surface and decays within ˜300 nm. Molecules in the bulk solution, at the distances larger than 300 nm are not excited. A 50 mW 488 nm laser at 10% power was used to excite oligonucleotides labeled with FAM. Images were collected by an Andor iXon EMCCD camera with a 100× lens and 100 ms exposure time. For each target, 100 (10 by 10 array) images were taken in ˜30 s. MATLAB software was used to analyze the images. The intensity was measured from each pixel of image (˜150 nm by 150 nm) for 100 images to generate the average fluorescence intensity.

The fluorescence intensity of the invented biochips listed in TABLE 3 show that there are almost the same fluorescence intensity measured by using the labelling moieties designed for two different locations of GPC1 mRNA (base location: 2,034 and 3,316).

TABLE 3

| | Base | Fluorescence | Standard |
|---|---|---|---|
| Biochip | location | Intensity (×10⁶) | derivation(s.d.) |
| LN-MB1 | 2,034 | 7.6 | 2.3 |
| LN-MB2 | 3,316 | 7.2 | 2.0 |
| LN-CHDC1 | 2,034 | 67.8 | 15.3 |
| LN-CHDC2 | 3,316 | 60.5 | 12.5 |
| LPHN-MB1 | 2,034 | 787.3 | 156.8 |
| LPHN-MB2 | 3,316 | 669.7 | 125.6 |
| LPHN-CHDC1 | 2,034 | 3876.1 | 295.2 |
| LPHN-CHDC2 | 3,316 | 3520.7 | 270.8 |

### Example 1

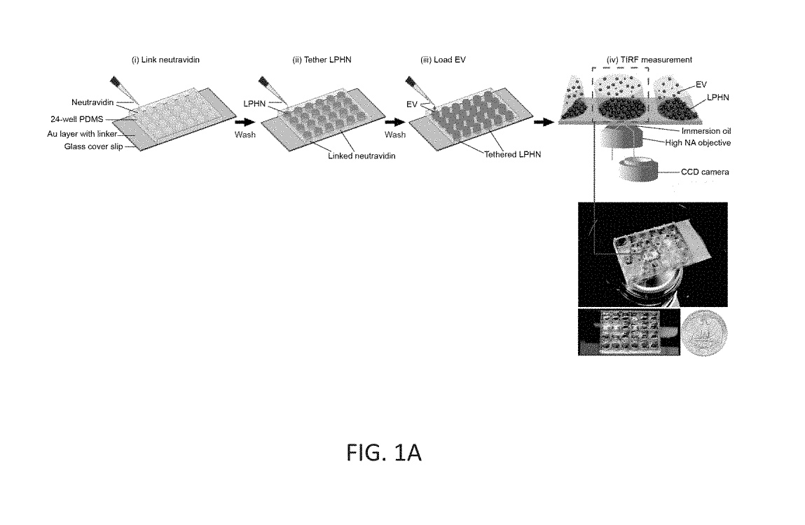

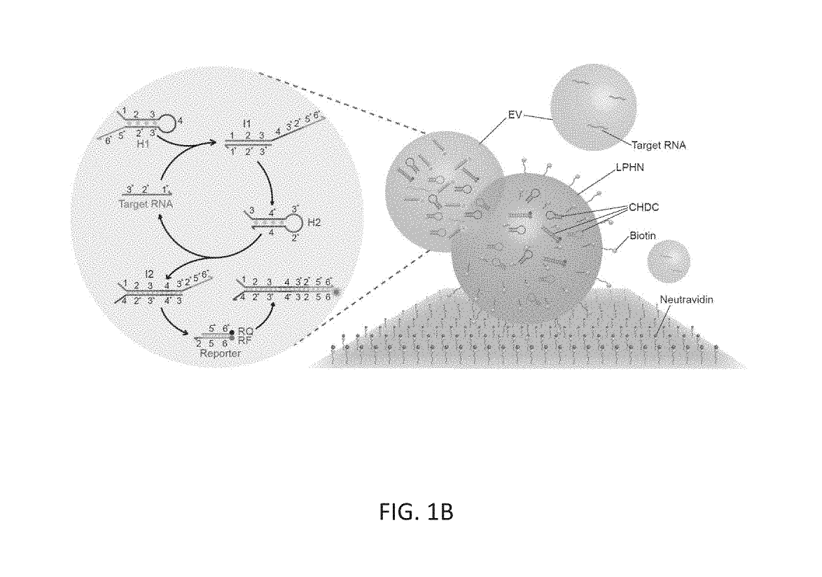

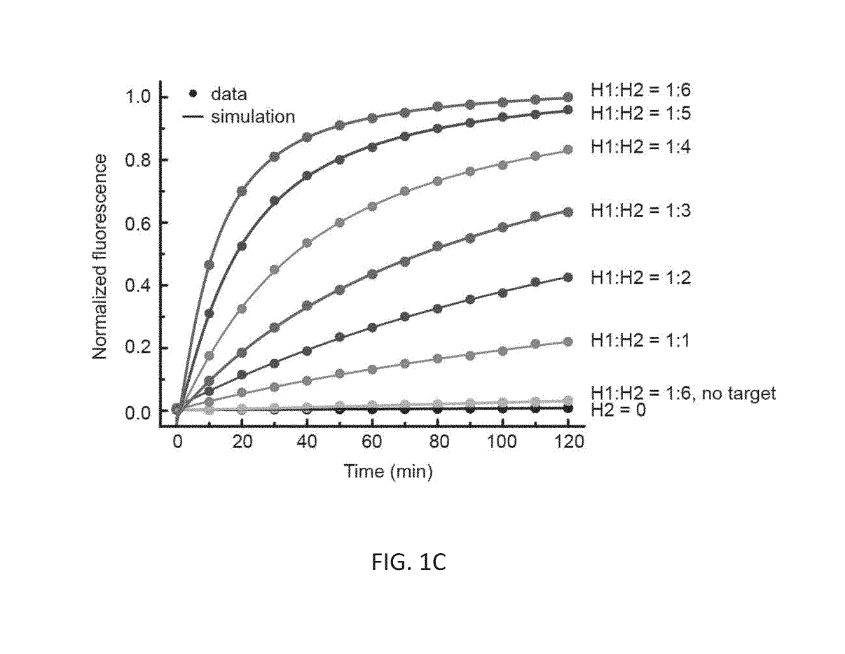

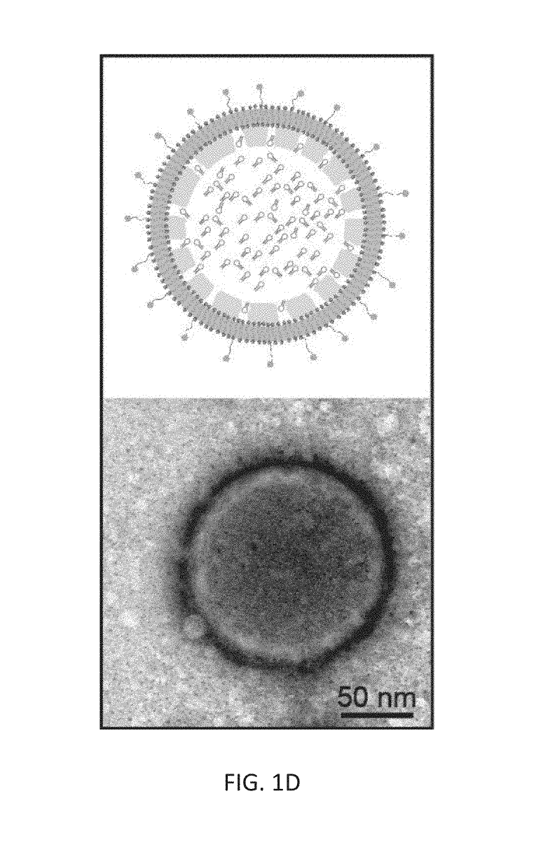

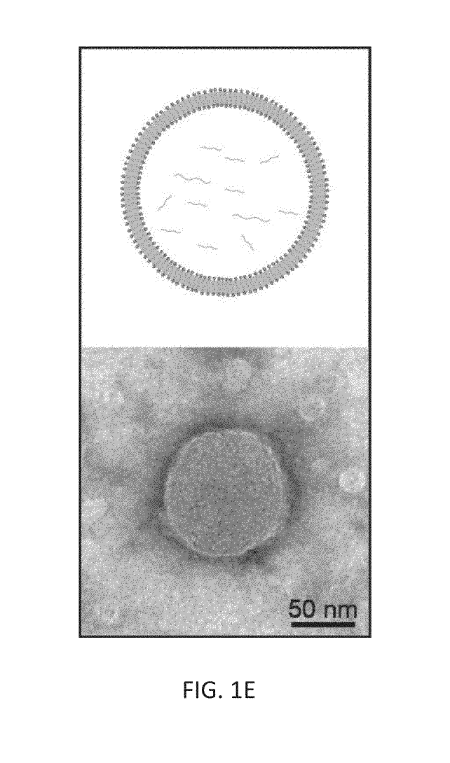

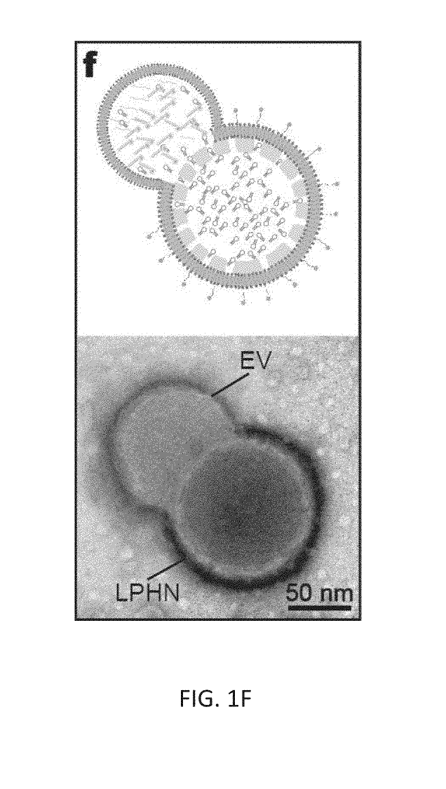

In this example, a model system is described which allows optimization of conditions for the LPHN-CHDC1 biochip. FIG. 1A shows an overall illustration of the smart system and how it works. As zoomed in FIG. 1B, specific CHDC1 consisting of H1, H2 and Reporter for GPC1 mRNA is encapsulated in LPHNs which are tethered on a chip through biotin-avidin interaction. Cationic LPHNs can capture negatively charged EVs by electrostatic interaction to form larger nanoscale complexes. The LPHN-EV fusion leads to mixing of H1, H2 and Reporter in the LPHN with target RNA in the EV. Consequently, the binding of target RNA to the exposed toehold domain 1 (red) of H1 would gradually initiate a strand displacement, generating an intermediate complex (I1) through domain hybridization (1-2-3 and 3*-2*-1*). The released toehold domain 3* in I1 further triggers branch migration on domain 3-4*-3*-2* of H2 to form the H1-H2 duplex (I2), followed by displacement of target RNA for the next catalytic round. Domain 2*-5*-6* on the I2 is fully complementary to Reporter-F (RF) that lights up inside EVs. Fluorescence signal of RF is observed by the total internal reflection fluorescence (TIRF) microscopy, which has high detection sensitivity upon near-interface (<300 nm). Therefore, the target RNA can trigger the hybridization between H1 and H2 for multiple cycles, and further denaturize the Reporter to provide signal amplification. Kinetics of catalyzed reactions was measured at varied H1:H2 ratios (1:1 to 1:6) with constant H1 and Reporter quantity (H1=Reporter=80 pmol). The results revealed elevated fluorescence intensity with the increasing H1:H2 ratio, and the optimized H1:H2 ratio (1:6) was chosen based on the reaction rate and EE of LPHN (FIG. 1C).

Structure characteristics of LPHN, EV and their fusion complex were depicted in FIG. 1D-1F. LPHN has a core-shell-corona structure, which exhibits three layers with different electron densities (FIG. 1D). The dark outer corona represents the stained DOTMA/Biotin-PEG-DSPE layer, the middle porous PLGA shell has a thickness of approximately 10-15 nm and inner hollow core contains CHDC1. PLGA-based particles fabricated by w₁/o/w₂ solvent evaporation technique could achieve well-defined porous hollow structure. Because of the nanoscaled diameter of LPHN, the porous channels within polymer shell are too small to be observed by TEM in this work. The hollow core of LPHN provides enough space for centralizing all components of CHDC1, such as H1, H2 and Reporter, required in the reaction circuit. Real EV typically displays lipid bilayer-enclosed structure (FIG. 1E). After outer lipid layer of LPHN fusing with EV, the pore canals within polymer shell provide the transport pathway for the mixing and hybridization of encapsulated. CHDC1 with EV-associated target RNAs (FIG. 1F). We thus hypothesize that CHDC1 circuitry can be well-performed in the LPHN-EV complex and fluorescence signal would be greatly enhanced without background increase. Conventional MB and cationic LN were also used for comparison with CHDC1 and LPHN, respectively. According to the selected region of human GPC1 mRNA (NCBI reference#: NM_002081.2), MB1 and CHDC1 sequences were rationally designed and encapsulated respectively in monodisperse LNs and LPHNs (LN-MB1, LN-CHDC1, LPHN-MB1 and LPHN-CHDC1) with the comparable diameter (˜100 nm), positive surface charge (˜30 mV) and EE (˜80%)

### Example 2

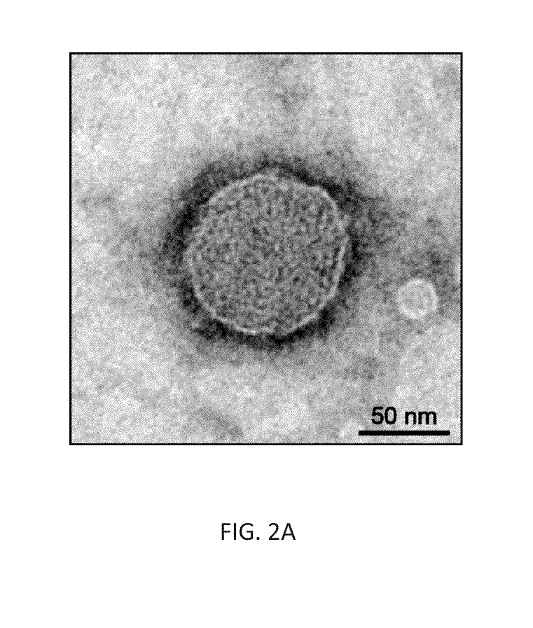

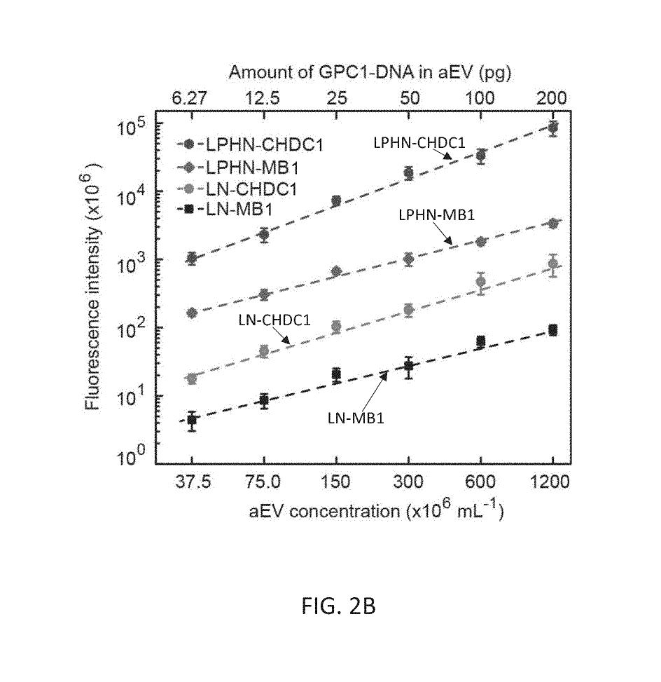

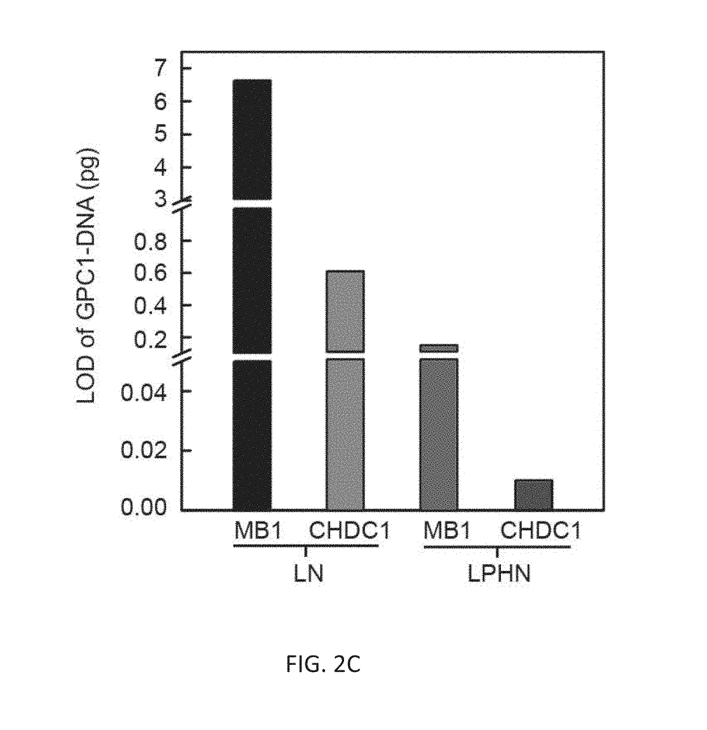

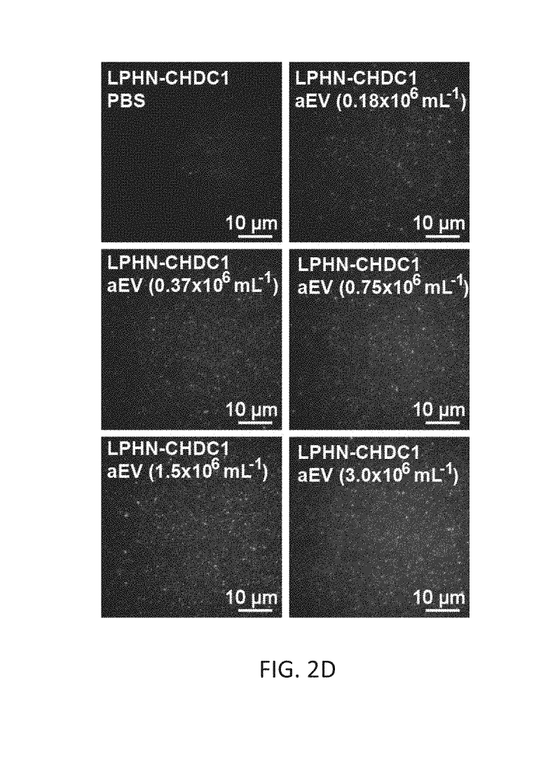

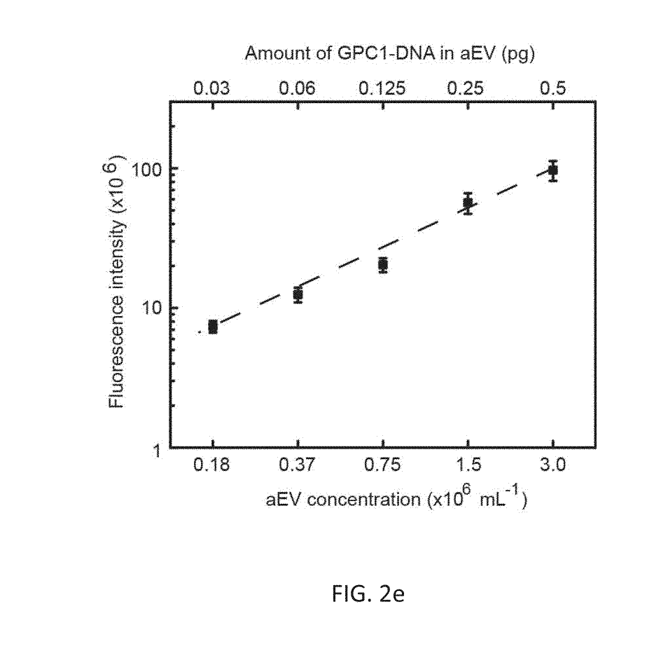

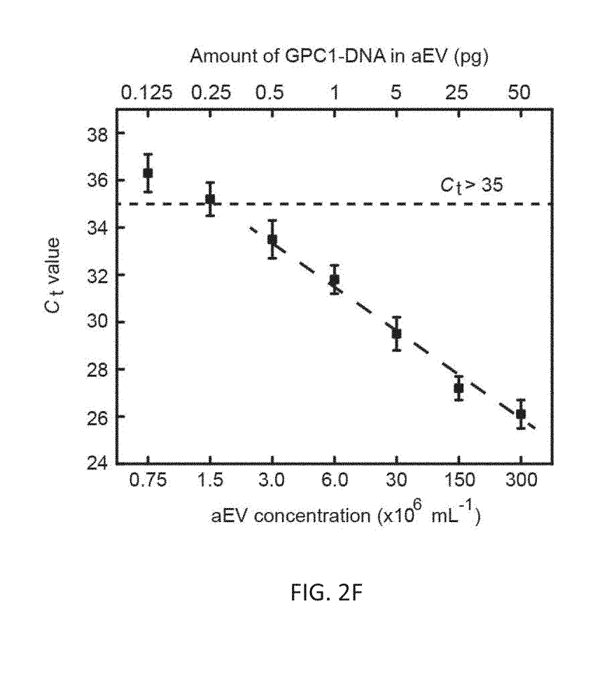

To develop a standard for biochip calibration, anionic lipoplex nanoparticles containing GPC1 DNA, termed artificial EVs (aEVs), were fabricated to mimic real EVs with the similar membrane structure. 50-150 nm diameter and slightly negative surface charge (−8.3 mV) (FIG. 2A). Since a target RNA in real EVs has a small copy number along with other RNAs, we prepared aEVs containing 1% of single strand GPC1 DNA mixed with 99% of low-cost miR54-DNA (scramble DNA) (molar ratio). The aEV concentration analyzed by Nanosight is 3.0×10¹⁰/mL and the calculated copy number of encapsulated GPC1 DNA was 270 strands per aEV. The fluorescence intensities of MB1 and CHDC1 respectively in the absence of target GPC1 DNA were firstly tested using aEV containing 100% of scramble DNA (aEV-SCR). Negligible fluorescence signal was observed in aEV-SCR similar in PBS as expected, which demonstrated our designed MB1 and CHDC1 were highly specific. Typical TIRF fluorescence images and linear calibration curves revealed fluorescence intensity of GPC1 DNA expression in aEVs using LN-MB1, LN-CHDC1, LPHN-MB1 or LPHN-CHDC1 biochip increased in proportion to the aEV concentration (1.2˜40% dilution equal to 37.5˜1200×10⁶/mL) (FIG. 2B). LN-CHDC1, LPHN-MB1 and LPHN-CHDC1 biochip showed fluorescence enhancement over LN-MB at each aEV concentration, particularly for LPHN-CHDC1/LN-MB1 biochip, reaching 236- and 914-fold at 1.2% and 40% of aEVs, respectively. The linearly extrapolated limit of detection (LOD) for GPC1 DNA was calculated to be 6.60 pg (298 amol), 0.6 pg (27.5 amol), 0.15 pg (6.88 amol) and 0.01 pg (0.46 amol) using LN-MB1 , LN-CHDC1, LPHN-MB1 and LPHN-CHDC1 biochip respectively based on the detection limit and encapsulation efficiency of aEVs (FIG. 2C). These results indicated enhanced catalytic amplification efficacy of CHDC1 over commonly used MB1, and LPHN was more suitable than LN for MB1/CHDC1 hybridizing with target RNAs in nanoparticle-EV fusion complex. In comparison to the core-shell-corona structure of LPHN, cationic LN typically displays a multilamellar (onion-like) structure in which negatively charged nucleic acids are sandwiched between cationic lipid bilayers. The, characteristic of onion-like structure was supposed to inhibit MB1/CHDC 1 encapsulated in the inner layers of LN to hybridize with target GPC1 DNAs in aEV when forming LN-aEV fusion complex. Therefore, the utilization of MB1/CHDC1 in LN is limited.

LPHN-CHDC1 biochip with the highest amplification capability has further been titrated with much lower concentrations of aEVs (0.18˜3.0×10⁶/mL) containing 0.03˜0.5 pg GPC1 DNA. Typical TIRF fluorescence images and linear calibration curves show that fluorescence intensity was aEV concentration-dependent, and calculated LOD of GPC1 DNA reached 0.01 pg for LPHN-CHDC1 biochip (FIG. 2D-2E). For comparison, quantitative PCR (qPCR) reaction was also performed using aEVs with low concentration range (0.75˜300×10⁶/mL, 0.125˜50 pg GPC1 DNA) (FIG. 2F). When aEV concentration was below 1.5×10⁶/mL (0.25 pg GPC1 DNA), the Cₜ value was over 35 and revealed non-linear correlation with aEV concentration (FIG. 2F).

### Example 3

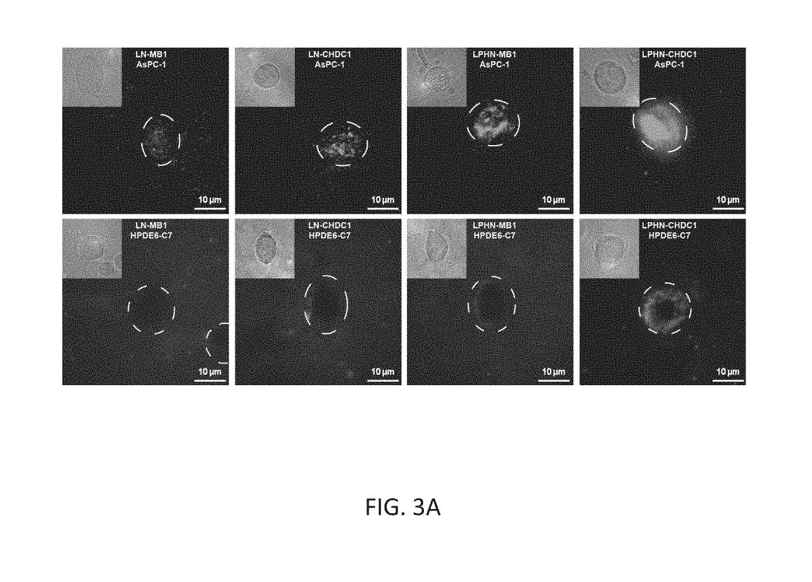

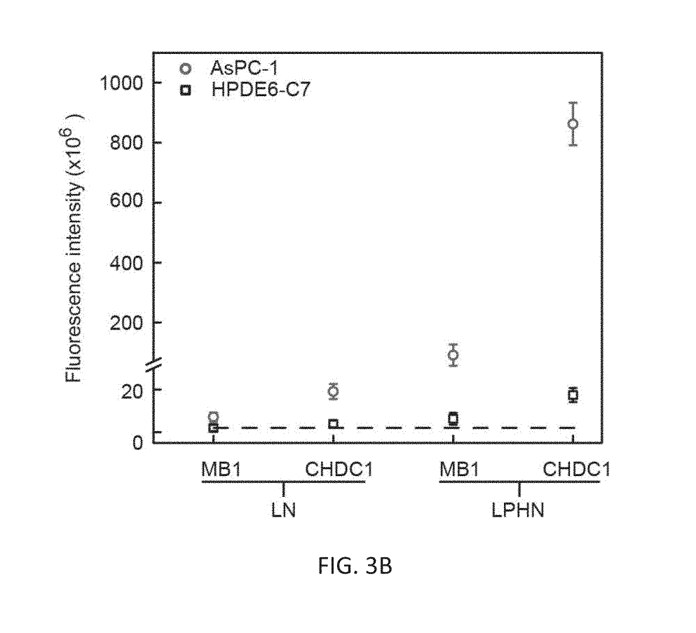

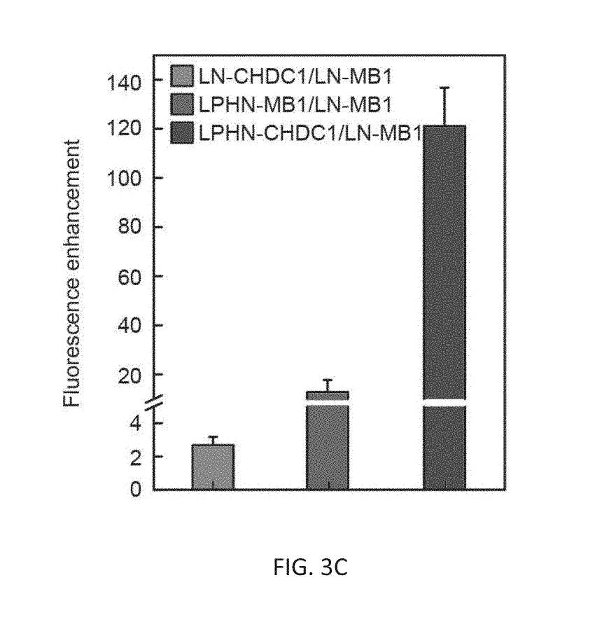

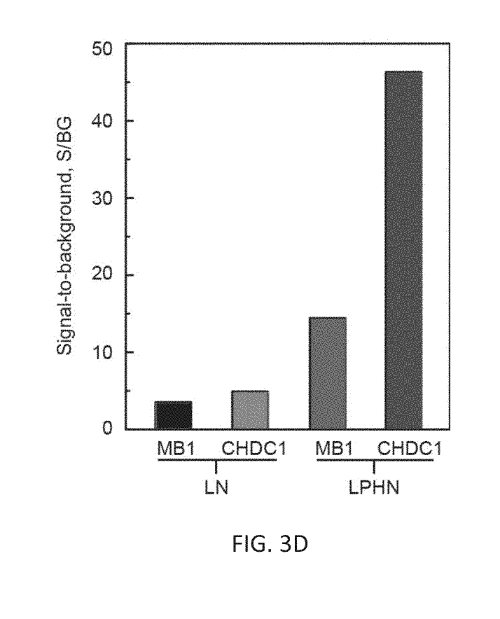

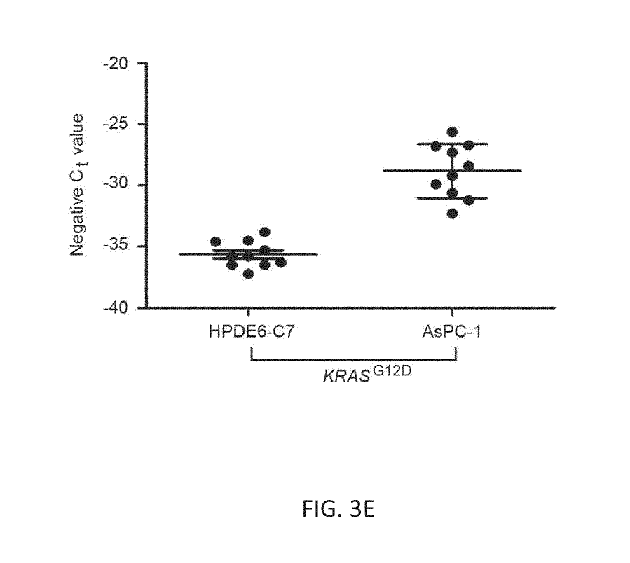

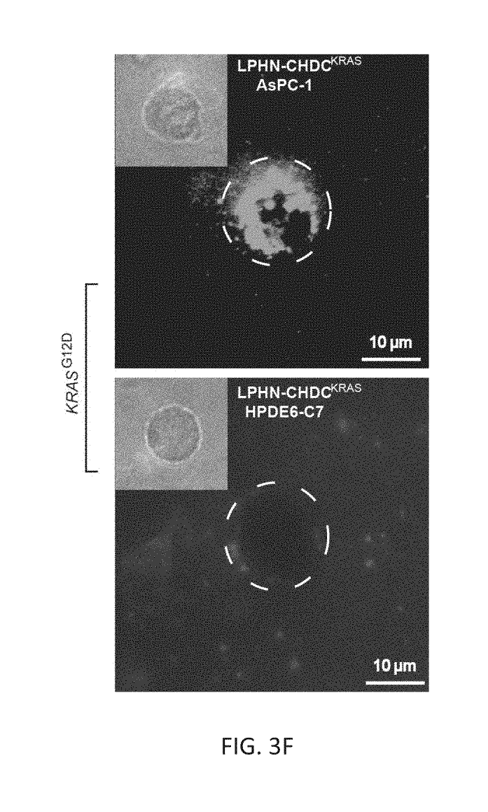

After internalized by living cells, the imaging capability and amplification effectiveness of LPHN-CHDC1 biochip was compared with LN-MB1, LN-CHDC1 and LPHN-MB1 biochips. High expression level of GPC1 mRNA was detected in pancreatic cancer cell lines (AsPC-1) compared to non-cancerous cells (HPDE6-C7) by qRT-PCR. The TIRF images showed that apparent fluorescence signals were observed in AsPC-1 cells, in contrast to the negligible or faint signals in HPDE6-C7 control cells (inside upper left figure was phase contrast image of each single cell) (FIG. 3A), which are consistent with the PCR results. Further quantitative analysis of image data showed the fluorescence intensity in AsPC-1 cells with LN-CHDC1, LPHN-MB 1 and LPHN-CHDC1 biochip were 2.6-, 12- and 121-fold higher than that with LN-MB1, respectively, while HPDE6-C7 cells exhibited the relative low fluorescence intensity levels (FIG. 3B-3C). The faint fluorescence detected in HPDE6-C7 cells was due to the low expression level of GPC1 mRNA, signal of which was amplified by LPHN-CHDC1 biochip, not non-specificity (FIG. 3A bottom right). The large increase of fluorescence intensity in AsPC-1 cells (signal) and only modest increase in HPDE6-C7 cells (background) resulted in the significant increase of signal-to-background (S/BG), especially for LPHN-CHDC1 biochip reaching 46-fold, which can well distinguish AsPC-1 cancer cells from HPDE6-C7 normal cells (FIG. 3D). The effectiveness of LPHN-CHDC biochip was further demonstrated by KRAS mutation detection. KRAS is a frequently mutated gene in pancreatic ductal adenocarcinoma (PDAC). qRT-PCR identified AsPC-1 cells with KRAS^{G12D} mutation, while HPDE6-C7 control cells did not exhibit KRAS^{G12D} mutation (FIG. 3E). A CHDC composition designed for KRAS^{G12D} mutation is list in TABLE 4 and the symbol of [A], [C], [G], and [T] represents a modified base of locked nucleic acid (LNA) of A, C, G and T, respectively. Both TIRF images and fluorescence microscopy images revealed an intense fluorescence signal of KRAS^{G12D} expression in AsPC-1 cells, in contrast to the negligible signal in HPDE6-C7 cells, indicating the excellent selectivity of LPHN-CHDC^{KRAS} for target KRAS^{G12D} in cancer cells (FIG. 3F). These results demonstrated that the invented LPHN-CHDC biochips could achieve the highest amplified imaging of specific mRNAs in living cells, distinguishing pancreatic cancer cells or even mutant cells from normal pancreatic cells.

TABLE 4

| Name | DNA sequence, listed 5′ to 3′ |
|---|---|
| CHDC^{KRAS}-H1 | ACG[C]CAT [C]AG[C]TC [C]AACT |
| | GCCCTGAGATTA AGTTGGAGCTG TCCACCTT |
| | CACCCTCA |
| CHDC^{KRAS}-H2 | CAACT TAATCTCAGGGC AGTTG GAGCTG |
| | GCCCTGAGATTA |
| CHDC^{KRAS}-RQ | TCCACCTT CACCCTCA /BHQ1/ |
| CHDC^{KRAS}-RF | /FAM/T[G]AG[G]GT[G] AA[G]GT[G]GA |
| | CAGCTC |

### Example 4

To further demonstrate the uniqueness of our lipid-polymer hybrid nanoparticles containing the components for performing catalyzed hairpin DNA circuit (LPHN-CHDC 1) biochip to quantify low concentration levels of EVs secreted from living cancer cells, the cell conditioned medium containing EVs secreted by AsPC-1 or HPDE6-C7 cells was directly applied to the LPHN-CHDC 1 biochip without EV isolation. NanoSight™ analysis revealed that the EV concentration was around 10⁸ mL⁻¹ in both AsPC-1 and HPDE6-C7 cell conditioned mediums. qRT-PCR analysis revealed a much higher expression level of GPC1 mRNA in AsPC-1 cells-derived EVs (AsPC-1 EVs) than in HPDE6-C7 cells-derived EVs (HPDE6-C7 EVs).

To ensure that our molecular beacons (MBs) and the biomolecules or components (polynucleotides) for performing catalyzed hairpin DNA circuits (CHDCs) can indeed detect the GPC1 mRNA fragments in EVs, we designed two probes to hybridize with different base locations of the GPC1 mRNA sequence (i.e. base locations 2,034 and 3,316) and tested the probe expression in AsPC-1 EVs. The very similar fluorescence signals between MB1 and MB2, and CHDC1 and CHDC2 in both LN and LPHN confirmed that the two designed MBs and CHDCs could target GPC1 mRNA or its fragments in cancer cell secreted EVs, even though the binding sites were different. These results imply that the MB/CHDC based detection of only a small sequence (˜20 bases) on the target mRNA and its fragments can represent well the presence of the entire mRNA in EVs.

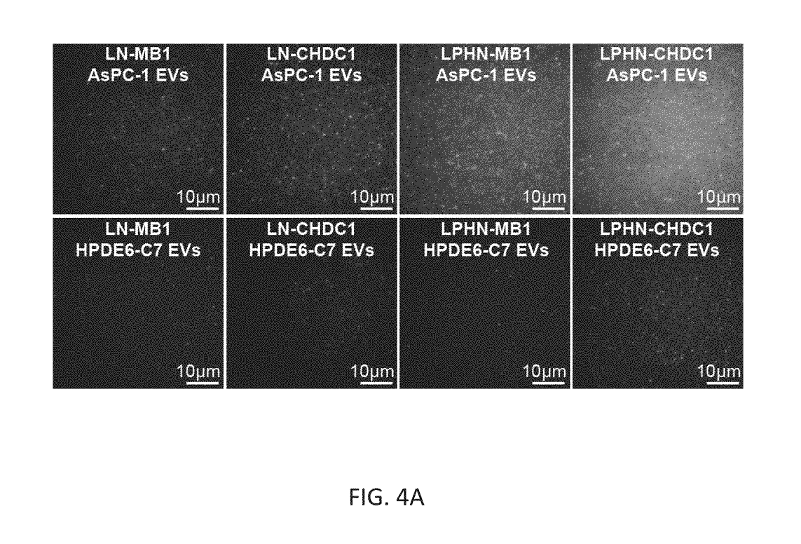

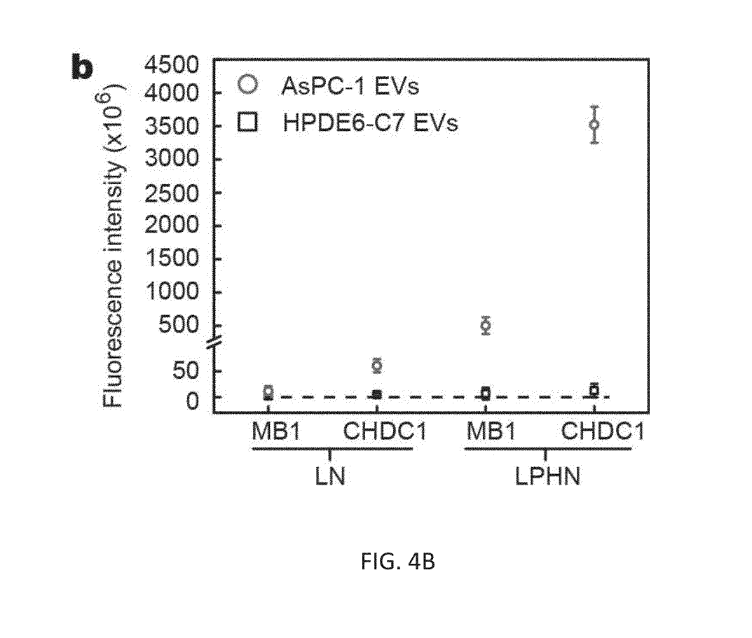

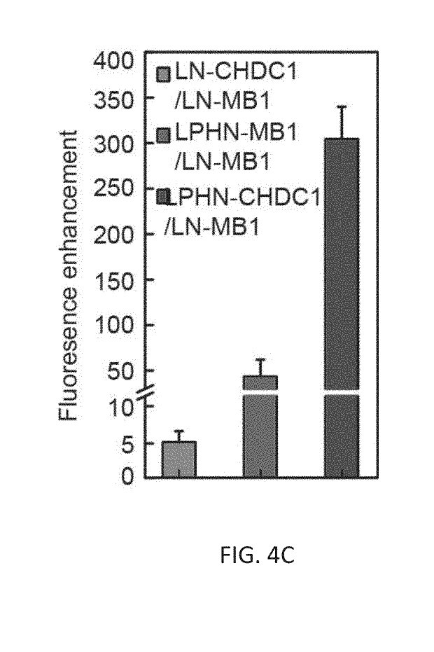

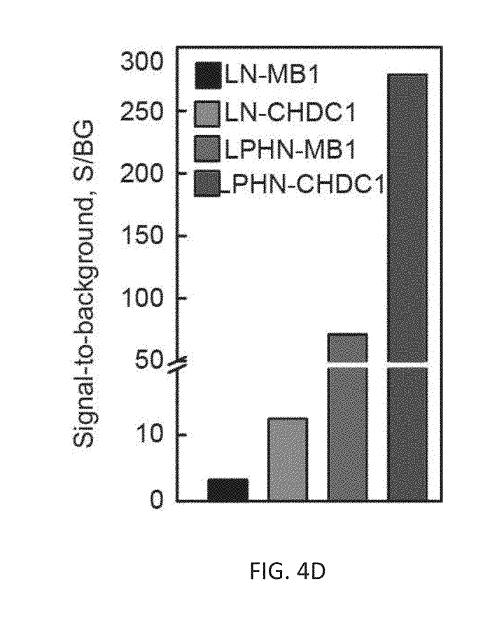

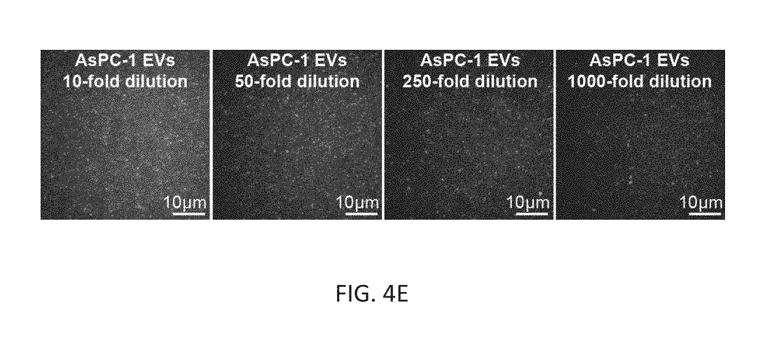

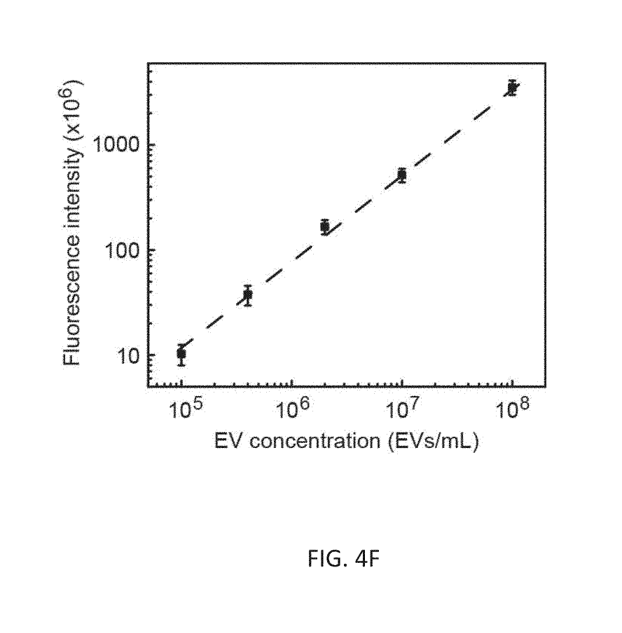

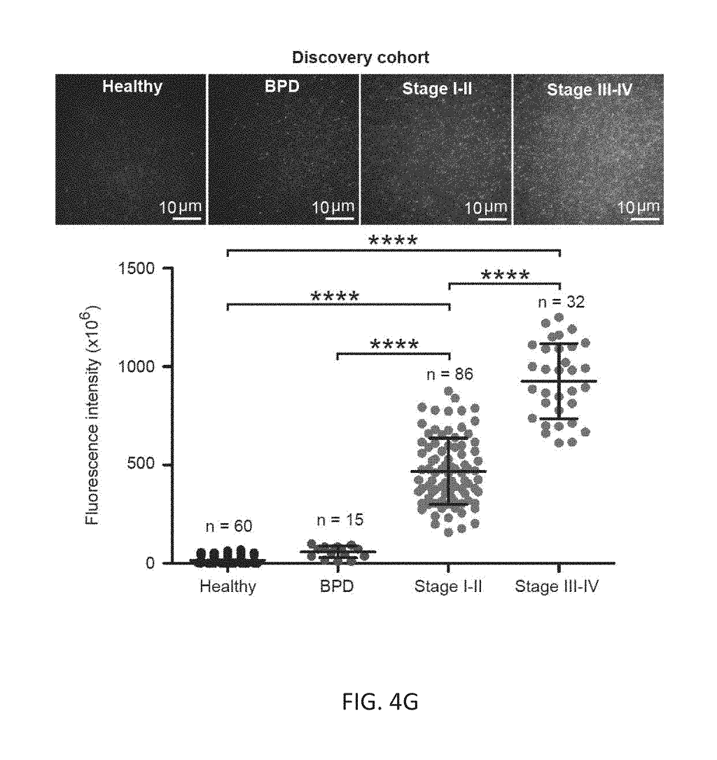

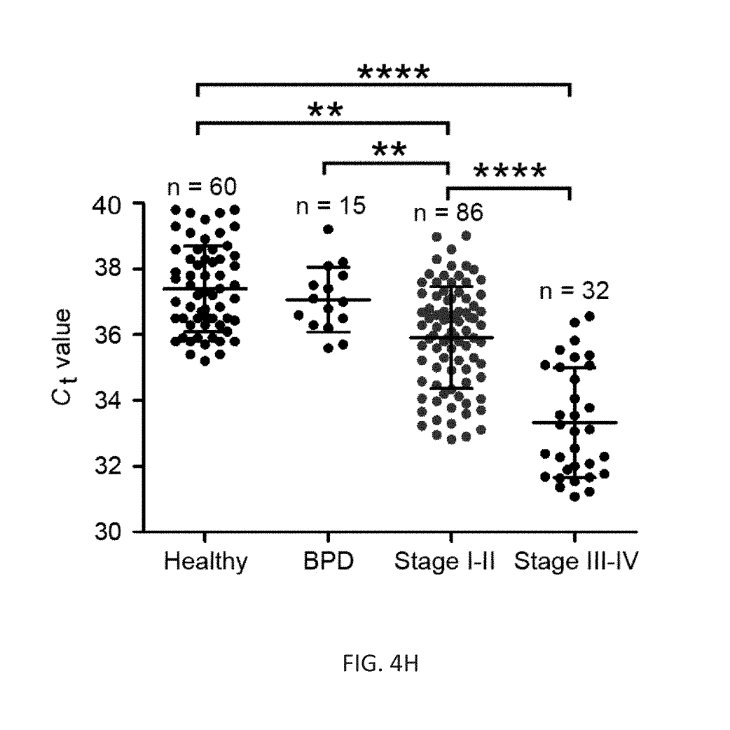

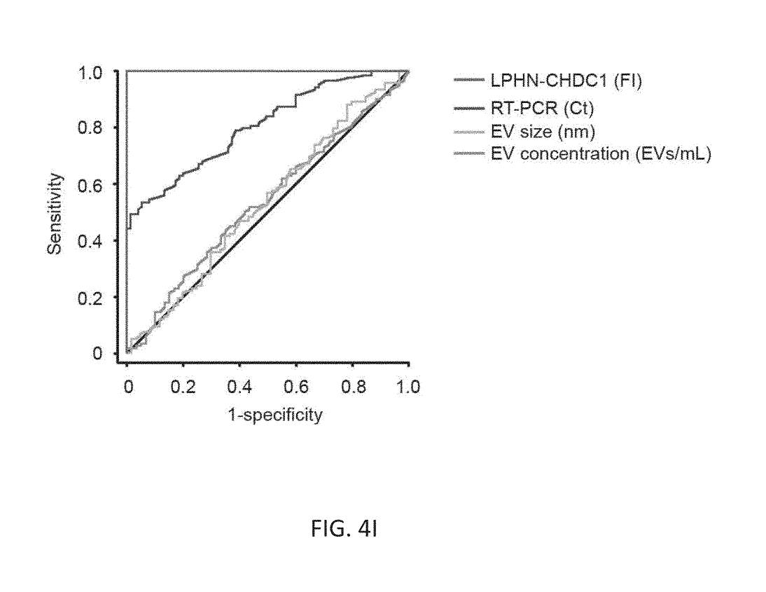

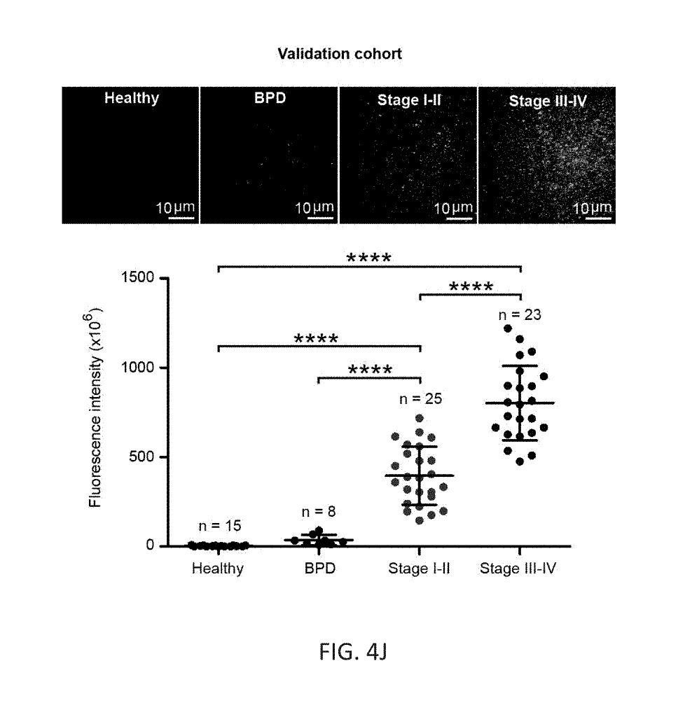

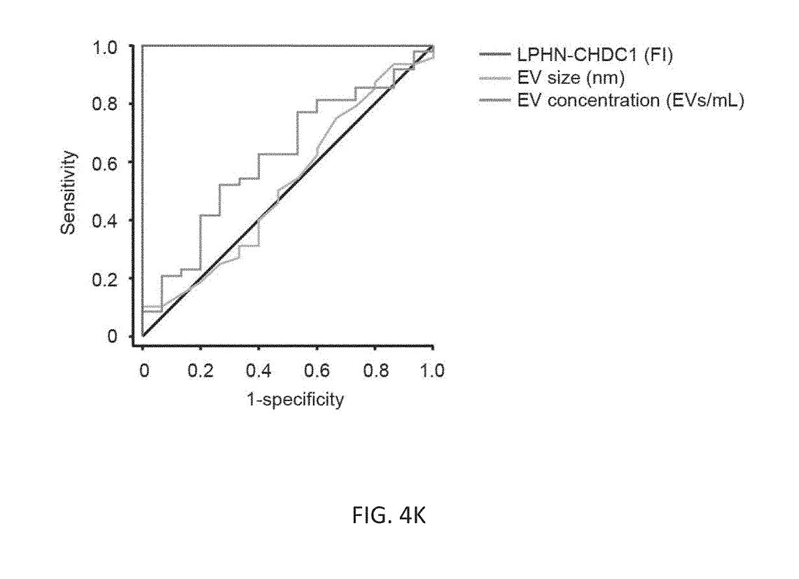

As expected, FIG. 4A shows much higher signals from AsPC-1 EVs compared to those from HPDE6-C7 EVs. Statistical analysis of image data revealed that the fluorescence intensity of the GPC1 mRNA expression in AsPC-1 EVs using lipoplex nanoparticles containing components for performing catalyzed hairpin DNA circuit (LN-CHDC1), lipid-polymer hybrid nanoparticles containing molecular beacon (LPHN-MB1), and LPHN-CHDC1 were 5.2-, 43- and 304-fold higher than that using lipoplex nanoparticles containing molecular beacon (LN-MB1), respectively, while HPDE6-C7 EVs exhibited a negligible fluorescence intensity (FIGS. 4B-4C). The significant difference in fluorescence intensity between AsPC-1 EVs and HPDE6-C7 EVs resulted in a large signal to background ratio, especially for LPHN-CHDC1 biochip, which reached 278-fold (FIG. 4D), indicating its high efficacy for the detection of cancer EVs. In our LPHN-CHDC assay, the signal to background ratio can be greatly enhanced by proper selection of the image cutoff level based on the background fluorescence. MATLAB software was used for analyzing the TIRF images. The intensity was measured at each pixel of the image for 100 images to generate an average fluorescence intensity. We selected a cutoff level for a higher signal to background ratio, which is not achievable by qRT-PCR. Besides, the high expression of target RNA would lead to a higher amplification rate and faster reaction rate in the CHDC amplification system, which consequently resulted in a larger difference fluorescence intensity. Furthermore, TIRF used for the fluorescence measurement our LPHN-CHDC1 biochip assay only allows the molecules very close to the surface (<300 nm) to be excited, while the fluorescence detector used in PCR measures the total fluorescence intensity from the whole sample solution which may add noise to the image. The sensitivity of LPHN-CHDC1 biochip was further verified based on 10- (˜10⁷ mL⁻¹), 50- (˜2×10⁶ mL⁻¹), 250- (˜4×10⁵ mL⁻¹) and 1000-fold (˜10⁵ mL⁻¹) dilution of AsPC-1 cells in the conditioned medium. Typical TIRF images and the linear calibration curve revealed that LPHN-CHDC1 biochip was able to detect EV levels as low as 10⁵ mL⁻¹, and the calculated LOD for AsPC-1 EVs was 57550 mL⁻¹ (˜60 EVs per μL) (FIGS. 4E-4F). For comparison, we also detected GPC1 mRNA expression in the cell conditioned medium after ultracentrifugation (i.e, supernatant) and recovered EV pellets collected at the bottom of the ultracentrifugation tube using LPHN biochips. The results show that the fluorescence signals of both LPHN-MB1 and LPHN-CHDC1 biochips increased somewhat by comparing the recovered EV pellets and EVs in the original cell conditioned medium because the EV concentration in the pellet was higher than that in the cell conditioned medium, while the fluorescence signal of supernatant after ultracentrifugation was very low. This experiment demonstrates that only RNA targets within EVs, not free RNAs, were detected by the invented LPHN biochip.

### Example 5

Finally, we evaluated GPC1 mRNA levels in human serum EVs from pancreatic ductal adenocarcinoma (PDAC) patients at stage I-II (n=86), stage III-IV (n=32), benign pancreatic disease (BPD, n=15; patients with pancreatitis), and healthy donors (n=60) in a discovery study. Serum samples were directly applied on the lipid-polymer hybrid nanoparticles containing catalyzed hairpin DNA circuit (LPHN-CHDC1) biochip without EV isolation. A comparison experiment between total serum and pre-isolated EVs was performed by using our LPHN-CHDC1 biochip. The results revealed relatively small difference in fluorescence signals between total serum and pre-isolated EVs and the trend among samples remained the same. This is because the concentration of EVs in human serum is over 10¹² EVs per mL, while the estimated maximum EV capture by the tethered nanoparticles in a single well (4 mm diameter) on the chip surface is ˜10⁹. We added 10 μL serum in each well, which contains >10¹⁰ EVs, a number much larger than the capacity needed to fuse with all tethered nanoparticles on our biochip. Therefore, pre-isolation of EVs from serum did not change the testing results much. TIRF analysis of discovery cohorts revealed that the fluorescence intensity of the GPC1 mRNA expression in serum EVs could effectively distinguish PDAC patients with stage I-IV from healthy donors and patients with BPD (P<0.0001; FIG. 4G). The BPD patients exhibited a similar EV GPC1 mRNA expression as healthy donors (FIG. 4G). We observed that all 86 PDAC patients with stage I-II exhibited higher levels of GPC1 mRNA expression than healthy donors and patients with BPD (P<0.0001) (FIG. 4G). Also, the GPC1 mRNA expression in EVs showed an upward trend between patients with stage I-II and stage III-IV (P<0.0001) (FIG. 4G). qRT-PCR data also revealed a difference of EV GPC1 mRNA expression between healthy donors and PDAC patients with stage III-IV (P<0.0001), however, there was a large signal overlap between healthy donors or BPD patients and PDAC patients with stage I-II (P<0.02; FIG. 4H). The main reason why qRT-PCR failed to distinguish early stage PDAC patients from healthy donors and BPD patients is as follows: For serum sample, circulating EVs are secreted by almost all mammalian cells, in which EVs secreted from cancer cells represent only a small fraction of the EV population especially in early-stage cancer. In most current EV RNA detection techniques including PCR-based methods, all EVs in the sample are lysed together for total RNA extraction regardless of their origins. As a result, dysregulated RNA targets in EVs secreted from cancer cells are mixed and highly diluted with the same RNAs in EVs secreted from non-cancer cells. Furthermore, mRNAs present in EVs, unlike in tissue and cells, are a mixture of intact and fragmented transcripts. The designed PCR primer pairs (length of primer ˜20 nucleotides) usually cannot duplicate small fragments (length of sequence<100 nucleotides) and recognize fragments without primer binding sites, which restricts the amplification process. In contrast, our biochip assay does not need EV isolation and RNA extraction/concentration. When lipid-polymer hybrid nanoparticles (LPHNs) fuse with EVs, the formed LPHN-EV nanoscale complex would prevent leakage of encapsulated target mRNAs. Moreover, the components or polynucleotides for performing catalyzed hairpin DNA circuit (CHDC) only hybridizes with around 20 nucleotides of a pre-specified RNA sequence, and thus is capable of detecting intact, large and small fragments of the mRNA target in EVs for much enhanced sensitivity. The ROC curve of LPHN-CHDC1 biochip showed an AUC of 1.0 in PDAC patients of stage I-IV compared to healthy donors and BPD patients, with a sensitivity and specificity of 100% (FIG. 4I). By contrast, qRT-PCR was inferior in classifying patients with PDAC from healthy donors and BPD patients (AUC=0.804) (FIG. 4I). Notably, neither the concentration of EVs nor their size was a valid parameter to distinguish PDAC patients from controls (FIG. 4I), consistently with Melo's results 26. A blind validation study was also carried out with patient samples from the same hospital. TIRF analysis of validation cohorts, composed of 25 patients with PDAC at stage I-II, 23 patients with PDAC at stage III-IV, 8 patients with BPD and 15 healthy donors, agreed well with the results of discovery cohorts (FIG. 4J). LPHN-CHDC biochip distinguished PDAC patients with stage I-IV from healthy donors and patients with BPD (FIG. 4J). The ROC curve of LPHN-CHDC1 biochip again showed an AUC of 1.0, and a specificity and sensitivity of 100% in each stage of pancreatic cancer, supporting its potential for early cancer detection (FIG. 4K). For further validation, we also conducted a blind test with patient samples collected from a different hospital. The results showed an AUC of 0.94. Although slightly less than the perfect detection results shown in FIG. 4, may be due to variations in patient diagnosis and sample collection procedures among different hospitals, this single EV GPC1 mRNA target can still serve as a very viable biomarker for PDAC diagnosis. We are currently conducting a larger scale multi-site validation study. To achieve long-term stability, the nanoparticles should be stored in a dried form. A stability and reproducibility comparison experiment of LPHN-CHDC 1 nanoparticles before and after lyophilization was performed. After lyophilization, our LPHN-CHDC 1 nanoparticles could maintain ˜87% signal recovery, indicating that lyophilization may extend the shelf-life of nanoparticles and make the assay much more robust and user friendly.

### Example 6

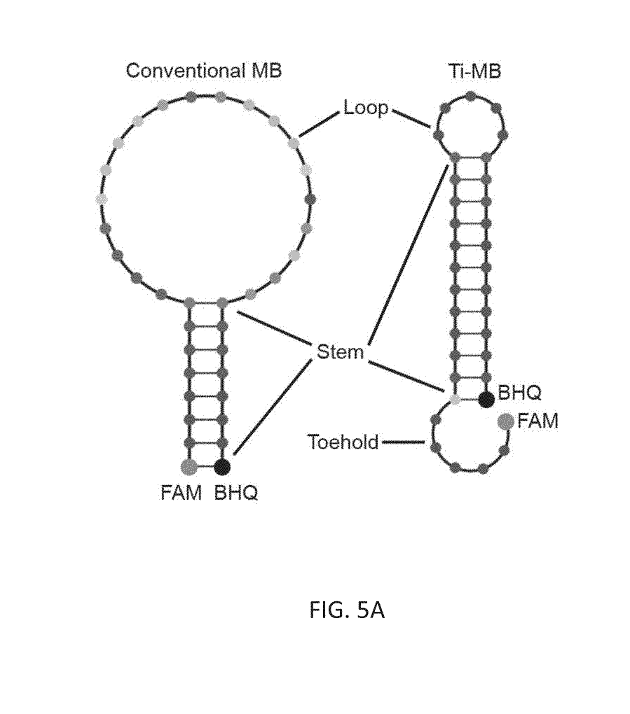

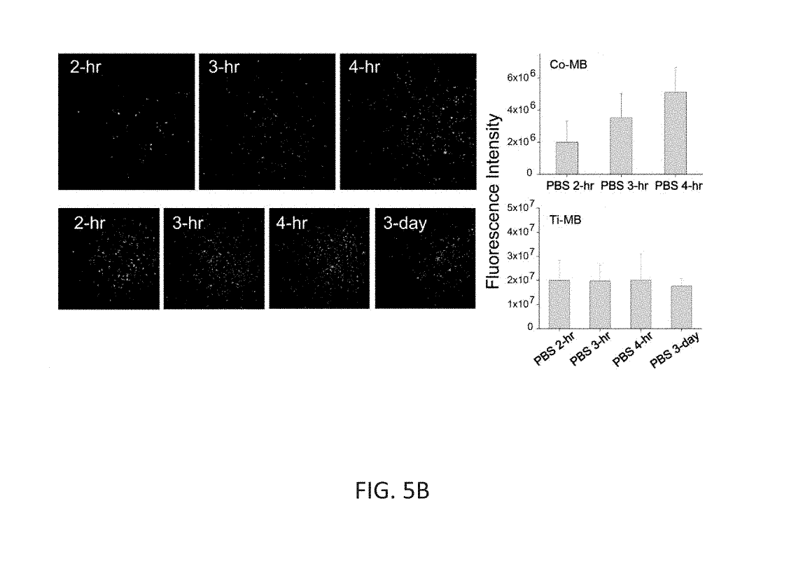

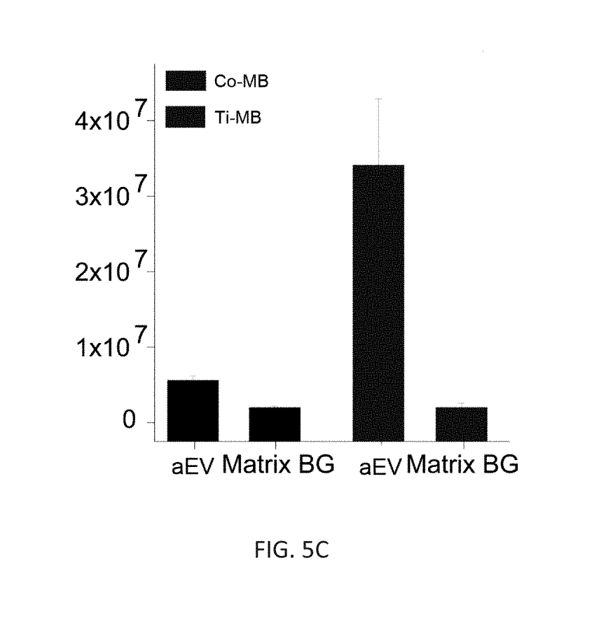

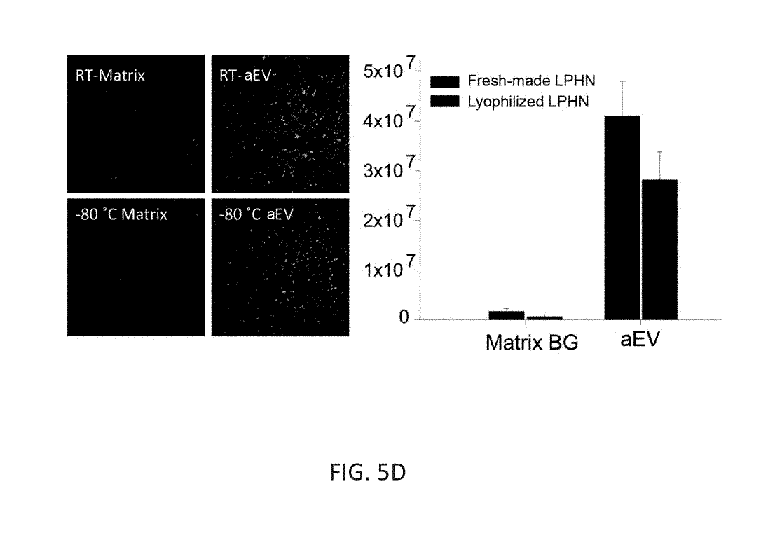

To demonstrate this design concept, we compared the performance of LPHN encapsulated with either the conventional MB (Co-MB) or the new designed Toehold-initiated (Ti-MB) against target artificial EVs containing miR-21. The structure of traditional MB and Ti-MB was shown in FIG. 5A. FIG. 5B shows that the new Ti-MBs encapsulated in LPHN could remain stable for >3 days in the liquid form. This is highly advantageous for preclinical applications where users often need to prepare their own nanoparticles with MBs for specific RNA targets.

Linear scale comparisons of background and target aEVs by using LPHN-Co-MB and LPHN-Ti-MB are shown in FIG. 5C. Using the Ti-MB, the signal was increased by 8-fold. In addition, the maximum achievable S/BG was improved by a factor of 20.

For clinical applications where RNA targets have been identified and validated as disease biomarkers, the users would like to use LPHN biochips with pre-synthesized LPHNs containing target MBs to simplify the assay. Lyophilization is a widely used method to prepare the dry form of nanoparticles for drug delivery. A comparison between a fresh-made LPHN and a lyophilized LPHN stored at −80° C. is shown in FIG. 5D using aEV-encapsulated with miR-21. The lyophilized LPHN was able to maintain ˜85-95% of the signal, which demonstrates the feasibility of lyophilization of LPHN.

It should be emphasized that the above-described embodiments of the present disclosure are merely possible examples of implementations, and are merely set forth for a clear understanding of the principles of the disclosure. Many variations and modifications may be made to the above-described embodiments. All such modifications and variations are intended to be included herein within the scope of this disclosure and protected by the following claims.

What is claimed is:

1. A lipid-polymer hybrid nanoparticle biochip, comprising a gold coating substrate with a surface layer on the gold coating and a nanoparticle, wherein the nanoparticle anchors on the surface layer and encapsulates labeling moiety which comprises molecular beacons (MB), Toehold-initiated molecular beacons (Ti-MB), biomolecules or components for performing catalyzed hairpin DNA circuit (CHDC), and quantum dots.

2. The lipid-polymer hybrid nanoparticle biochip of claim 1, wherein the surface layer being a self-assembly monolayer selected from the group consisting of 2-mercaptoethanol (βME), 6-mercaptohexanol, Biotin-PEG-thiol (HS-PEG-Biotin), thiol-backfiller molecules and combinations thereof.

3. The lipid-polymer hybrid nanoparticle biochip of claim 1, wherein surface of the nanoparticle further functionalizes with one comprises avidin-biotin, fluorescein-anti-FITC, hapten linkages of antibody molecules, peptides, carbohydrate, DNA and RNA.

4. The lipid-polymer hybrid nanoparticle biochip of claim 1, wherein the nanoparticle is formed by a polymer and a lipid, wherein the polymer comprises Polyethylenimine (PEI), Poly-L-Lysine (PLL), Poly-D-Lysine (PDL), Poly (ε-caprolactone) (PCL), Polylactide (PLA) and Poly (Lacitide-co-Glycolide) (PLGA), and wherein the lipid comprises 1,2-di-O-octadecenyl-3-trimethylammonium propane (DOTMA), 1,2-dioleoyl-3-trimethylammonium-propane (DOTAP), 3β-[N-(N′,N′-dimethylaminoethane)-carbamoyl]cholesterol hydrochloride (DC-Cholesterol), ionizable lipids, 1,2-di-O-octadecenyl-3-dimethylammonium propane (DODMA), 1,2-dioleoyl-3-dimethylammonium-propane (DODAP), non-ionizable lipids, L-α-phosphatidylcholine (EggPC, SoyPC), Cholesterol, 1,2-dipalmitoyl-sn-glycero-3-phosphocholine (DPPC), saturated fatty acid, 1,2-dioleoyl-sn-glycero-3-phosphocholine (DOPC), 1,2-distearoyl-sn-glycero-3-phosphoethanolamine-N-[methoxy(polyethylene glycol)] (DSPE-PEG), and PEG phospholipids.

5. The lipid-polymer hybrid nanoparticle biochip of claim 1, wherein the molecular beacons comprise a oligonucleotide, and the oligonucleotide has a fluorophore at 5′ end and a quencher at 3′ end.

6. The lipid-polymer hybrid nanoparticle biochip of claim 5, wherein the oligonucleotide is selected from the group consisting of SEQ ID NO: 1 and SEQ ID NO: 2.

7. The lipid-polymer hybrid nanoparticle biochip of claim 5, wherein the fluorophore at the 5′ end comprises FAM, TET, HEX, Cyanine dyes, TMR, ROX, JOE, Texas red, LC red 640, and LC red 705.

8. The lipid-polymer hybrid nanoparticle biochip of claim 5, wherein the quencher at the 3′ end comprises Black Hole Quenchers, Deep Dark Quenchers, QSY-7, QSY-21, Dabcyl, Eclipse, Iowa Black FQ, and Iowa Black RQ.

9. The lipid-polymer hybrid nanoparticle biochip of claim 1, wherein the Ti-MB comprises a oligonucleotide, wherein the oligonucleotide has a stem of 5 to 50 base pairs, a loop of 1 to 100 bases, and a toehold domain of 1 to 50 complementary bases to target DNA, RNA or the combination is added at the end of the stem, and wherein the oligonucleotide has a fluorophore at the 5′ end and a quencher at the 3′ end.

10. The lipid-polymer hybrid nanoparticle biochip of claim 1, wherein the biomolecules or components for performing catalyzed hairpin DNA circuit (CHDC) comprise a first hairpin DNA, a second hairpin DNA, a fluorophore-labeled oligonucleotide strand (RF) has 5 to 100 bases and a quencher-labeled oligonucleotide strand (RQ) has 5 to 100 bases complementary to the fluorophore-labeled oligonucleotide strand;
wherein the first hairpin DNA comprises a stem of 5 to 50 base pairs and a loop of 1 to 100 bases, a toehold domain of 1 to 50 complementary bases to a target DNA/RNA is added at the end of the stem and the second hairpin DNA comprises a stem of 5 to 50 base pairs and a loop of 1 to 100 bases, a toehold domain of 1 to 50 complementary bases to domain of the first hairpin DNA, and the first hairpin DNA and the second hairpin DNA form a duplex.

11. The lipid-polymer hybrid nanoparticle biochip of claim 10, wherein the first hairpin DNA is SEQ ID NO: 3, the second hairpin DNA is SEQ ID NO: 4, the quencher-labeled oligonucleotide strand (RQ) is SEQ ID NO: 5 having BHQ1 at the 3′ end and the fluorophore-labeled oligonucleotide strand (RF) is SEQ ID NO: 6 having FAM at the 5′ end.

12. The lipid-polymer hybrid nanoparticle biochip of claim 10, wherein the first hairpin DNA is SEQ ID NO: 7, the second hairpin DNA is SEQ ID NO: 8, the quencher-labeled oligonucleotide strand (RQ) is SEQ ID NO: 9 having BHQ1 at the 3′ end and the fluorophore-labeled oligonucleotide strand (RF) is SEQ ID NO:10 having FAM at the 5′ end.

13. The lipid-polymer hybrid nanoparticle biochip of claim 1, being applied to capture extracellular vesicle (EV), virus or cell for detecting one comprises DNA, RNA and protein.

14. A method of detecting the presence of a disease or condition in a subject comprising:
(1) providing a biological sample obtained from a subject;
(2) contacting the lipid polymer hybrid nanoparticle chip of claim 1 with the biological sample from the subject; and
(3) detecting the target intracellular RNA, DNA, proteins or the combinations existed in the biological sample from the subject by excitation level of a label of labeling moiety that occurs through the capture and incorporation of one comprises cells, cell secreted extracellular vesicles, virus, bacteria, and antigen that corresponds to a disease or condition.

15. The method of claim 14, wherein the labeling moiety comprises molecular beacons, toehold initiated molecular beacons (Ti-MB), biomolecules or components for performing catalyzed hairpin DNA circuit (CHDC) and quantum dots.

16. The method of claim 15, wherein the molecular beacon is oligonucleotide selected from the group consisting of SEQ ID NO: 1 and SEQ ID NO: 2, and the oligonucleotide has a fluorophore at the 5′ end and a quencher at the 3′ end.

17. The method of claim 15, wherein the biomolecules or components for performing catalyzed hairpin DNA circuit (CHDC) are selected from the group consisting of a first polynucleotide composition and a second polynucleotide composition, wherein the first polynucleotide composition comprises SEQ ID NO: 3, SEQ ID NO: 4, SEQ ID NO: 5 having BHQ1 at the 3′ end, and SEQ ID NO: 6 having FAM at the 5′ end and the second polynucleotide composition comprises SEQ ID NO: 7, SEQ ID NO: 8, SEQ ID NO: 9 having BHQ1 at the 3′ end, and SEQ ID NO:10 having FAM at the 5′ end.

18. The method of claim 14, wherein the biological sample from the subject comprises blood serum, blood plasma, whole blood, nasal aspirates, saliva, urine, sputum, feces, cell lysate, dialysis sampling, tissue biopsy, cell media, and a combination thereof.

19. The method of claim 14, wherein the disease or condition is selected from the group consisting of: cancer, viral infection, bacterial infection, heart attack, stroke, rhabdomyolysis, fertility, diabetes, obesity, metabolic syndrome, sepsis, inflammatory response, food safety, tuberculosis, and a combination thereof.

20. The method of claims 19, wherein the cancer comprises lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colon-rectal cancers, prostatic cancer, pancreatic cancer, and cancer cachexia.
